(19) United States
(12) Patent Application Publication
(10) Pub. No.: US 2023/0218608 A1
(43) Pub. Date: Jul. 13, 2023

(54) NEW STRATEGY FOR TREATING PANCREATIC CANCER

(71) Applicants: INSERM (INSTITUT NATIONAL DE LA SANTÉ ET DE LA RECHERCHE MÉDICALE), Paris (FR); UNIVERSITÉ D'AIX MARSEILLE, Marseille Cedex 07 (FR); CENTRE NATIONAL DE LA RECHERCHE SCIENTIFIQUE (CNRS), Paris (FR); INSTITUT JEAN PAOLI & IRENE CALMETTES, Marseille (FR)

(72) Inventors: Alice CARRIER, Marseille Cedex 9 (FR); Gabriela REYES CASTELLANOS, Marseille Cedex 9 (FR); Rawand MASOUD, Marseille (FR)

(21) Appl. No.: 18/001,616

(22) PCT Filed: Jun. 17, 2021
(86) PCT No.: PCT/EP2021/066507

§ 371 Date: Dec. 13, 2022

(30) Foreign Application Priority Data
Jun. 18, 2020 (EP) 20305666.8

(51) Int. Cl. A61K 31/495; A61P 35/00; A61P 1/18; A61K 31/7068; G01N 33/50; A61K 31/336; A61K 31/4458

(52) U.S. Cl. CPC A61K 31/495; A61P 35/00; A61P 1/18; A61K 31/7068; G01N 33/5011; A61K 31/336; A61K 31/4458; G01N 2800/52

(57) ABSTRACT

The present invention relates to the treatment of pancreatic cancer. In this study, the results of the inventors led them to highlight the non-explored but relevant pathway in the pancreatic cancer field, the Fatty Acid Oxidation (FAO) pathway. Interestingly, they found that the mitochondrial respiration of PDAC cells depends mostly on this pathway. Thus they hypothesized that inhibition of FAO could be an effective therapeutic strategy against PDAC. 10 Their data support the hypothesis that this metabolic pathway plays a crucial role in PDAC, as it has been reported in other types of cancer. Thus, the invention relates to an inhibitor of fatty acid oxidation (FAO) for use in the treatment of pancreatic cancer in a patient in need thereof.

### FIELD OF THE INVENTION

The present invention relates to an inhibitor of fatty acid oxidation (FAO) for use in the treatment of pancreatic cancer in a patient in need thereof.

### BACKGROUND OF THE INVENTION

Pancreatic Ductal AdenoCarcinoma (PDAC), the most common form of pancreatic cancer, remains an incurable disease with a rising incidence in developed countries. PDAC is the 11th most frequent cancer worldwide and the 7th leading cause of deaths in Europe and North America [1-3]. A major concern is that PDAC is predicted to become the second cause of cancer-related deaths in United States by 2030, surpassing breast and colorectal cancers [4-6]. In contrast with other types of cancer, the 5-year overall survival (OS) of patients with PDAC has improved from 2.5% in 1970-1977 to only 8% in 2007-2013. Even so, the 5-year OS in some countries is as low as 2% [2, 7].

There are several reasons to explain the poor outcome of PDAC. First, PDAC lacks early and specific symptoms, thus 80-90% of patients are diagnosed at advanced stage (unresectable or metastatic tumors) [8]. Then in these patients, the standard of care with cytotoxic therapies as Gemcitabine plus Nab-Paclitaxel provides only a modest increase in survival (in the range of weeks to months) [9, 10]. In the case of resectable tumors, the treatment with mFOLFIRINOX has been demonstrated to provide the longest median OS (54 months) [11]. However, most patients face chemoresistance and will develop advanced disease at some point during treatment [12]. So far, there is still no definite cure in patients with advanced-stage PDAC.

Further research is needed to better understand this disease, in order to develop novel and effective therapeutic strategies. Among these strategies, therapeutic agents targeting the tumor metabolism have shown promise and are likely to have a role in the future management of patients with PDAC [12-16]. Metabolic reprogramming is a hallmark of cancer that was first observed by Otto Warburg in 1924 (“Warburg effect”) [17, 18]. This feature refers to the ability of cancer cells to switch their metabolism in response to an increased energy demand required to support the anabolic reactions necessary for tumor growth, especially if this occurs in a hostile and nutrient-poor microenvironment. Particularly, PDAC possesses one of the most abundant stromal compartments of any tumor type, and this peculiar stroma is a source of both physical and oxidative stress that drives metabolic rewiring [19-21]. In this context, mitochondria are hubs for metabolic reactions and drive this reprogramming through multiple mechanisms [22]. In consequence, mitochondrial energetic metabolism is a current target in cancer therapy [23-29].

Mitochondria are dynamic cytoplasmic organelles playing a crucial role in cell physiology. Mitochondrial respiration produces approximately 90% of the cellular energy in the form of ATP, by oxidative phosphorylation (OXPHOS). The oxidation of three critical nutrients (glucose, lipids, and amino acids) fuels the TriCarboxylic Acid (TCA) cycle in the mitochondrial matrix. The TCA generates NADH and FADH₂, which provide electrons to the mitochondrial Electron Transport Chain (ETC) complexes in the inner membrane, creating a proton force used for high-efficiency ATP generation by the ATP-synthase (Complex V) [30]. Besides energy production, mitochondria exert other critical functions, including macromolecule biosynthesis and the generation of most of the cellular Reactive Oxygen Species (ROS). Moreover, mitochondria regulate redox status, calcium cytosolic concentrations, and cell death via intrinsic apoptosis [31].

In consequence, the multifaceted mitochondrial functions place mitochondria as key actors in cancer. Accordingly, accumulating evidence recognize that mitochondria play a central role in tumorigenesis, cancer progression, and resistance to chemotherapy. For instance, Tan et al [32] demonstrate that cancer cells devoid of mitochondrial DNA (mtDNA) exhibit delayed tumor growth. Another study showed that inhibition of mitochondrial protein synthesis combined with Gemcitabine decreases pancreatic cancer cell survival by two means: reducing cell proliferation (by ATP depletion) and enhancing apoptosis by Gemcitabine treatment (decreasing the mitochondrial inner membrane potential and increasing ROS production) [24]. Furthermore, mitochondrial metabolism was shown to be involved in resistance to therapies, and it has been proven that cancer cells lacking mtDNA or with low copy numbers are much more sensitive to cytotoxic drugs [33-35]. Particularly, pancreatic cancer resistance to Gemcitabine has been shown to be dependent on mitochondria-mediated apoptosis [36].

Because of signaling and metabolic alterations, cancer cells are inherently under increased oxidative stress. However, even in the presence of oxidative stressors (e.g. chemotherapy), cancer cells are able to keep balanced ROS levels by the production of antioxidants within a window that stimulates proliferation without causing cytotoxicity. Altogether, these findings demonstrate that mitochondria are critical for all facets of cancer progression, being central in the regulation of cell proliferation (by bioenergetics and biosynthesis), cell death (via apoptosis), and ROS production.

Of major importance, mitochondrial functions are targetable in clinical applications (diagnostic, prognostic, and therapy). In particular, mitochondrial oxidative phosphorylation (OXPHOS) is an emerging target in cancer therapy [25, 28, 29]. In this context, our team previously demonstrated that the OXPHOS rate in PDAC tumors is highly heterogeneous between patients, highlighting the need for personalized medicine (Masoud et al, in revision). Furthermore, this study showed that tumors with High OXPHOS activity are responsive to the targeting of mitochondrial Complex I (using the biguanide Phenformin) in combination with standard chemotherapy (Gemcitabine) (Masoud et al, in revision). More importantly, our results led us to highlight a non-explored but relevant pathway in the pancreatic cancer field, the Fatty Acid Oxidation (FAO) pathway.

In addition to glucose and amino acids, fatty acids are an extremely relevant energy source. Fatty acids are catabolized by the FAO pathway within the mitochondria. FAO comprises a cyclical series of reactions that result in the shortening of fatty acid molecules (beta-oxidation) to produce Acetyl-CoA and NADH/FADH₂. The generated Acetyl-CoA enters the mitochondrial TCA cycle, where it is further oxidized to generate NADH and FADH₂. Finally, the NADH and FADH₂ produced by both beta-oxidation and the TCA cycle are used by the mitochondrial respiratory chain to produce ATP. The carnitine palmitoyltransferase 1 (CPT1) is considered the rate-limiting enzyme in FAO, since it conjugates fatty acids with carnitine to translocate them into the mitochondria, where the acylcarnitines undergo FAO [37].

The pharmacological blockade of FAO has been pursued for the treatment of heart diseases. Consequently, FAO inhibitors have been approved for human use [37-41]. These drugs are used to inhibit degradation of lipids for energy production, and thereby to promote more oxygen-efficient utilization of glucose as an energy source in chronic ischemic cardiomyopathy [42]. CPT1 is a key enzyme in the FAO pathway, and can be pharmacologically targeted by drugs like Etomoxir and Perhexiline. In this work, we used Perhexiline and showed enhancement of the antitumoral effect of chemotherapy (Gemcitabine) resulting in a complete tumoral regression.

As most of tumor metabolism researchers are focused on glycolysis, glutaminolysis, and fatty acid synthesis, the importance of FAO in cancer has not been carefully examined, and its relevance has remained obscure [37].

### SUMMARY OF THE INVENTION

In this study, the results of the inventors led them to highlight the non-explored but relevant pathway in the pancreatic cancer field, the Fatty Acid Oxidation (FAO) pathway. Interestingly, they found that the mitochondrial respiration of PDAC cells depends mostly on this pathway. Thus they hypothesized that inhibition of FAO could be an effective therapeutic strategy against PDAC. Their data support the hypothesis that this metabolic pathway plays a crucial role in PDAC, as it has been reported in other types of cancer [43-52].

Thus, the present invention relates to an inhibitor of fatty acid oxidation (FAO) for use in the treatment of pancreatic cancer in a patient in need thereof. Particularly, the invention is defined by its claims.

### DETAILED DESCRIPTION OF THE INVENTION

### Therapeutic Method

A first object of the invention relates to an inhibitor of fatty acid oxidation (FAO) for use in the treatment of pancreatic cancer in a patient in need thereof.

The invention also relates to a method for treating a pancreatic cancer in a patient in need thereof by administrating to said patient an inhibitor of fatty acid oxidation (FAO).

As used herein, the term “Fatty Acid Oxidation (FAO)”, also known as beta-oxidation, has its general meaning in the art and denotes the major metabolic pathway that is responsible for the mitochondrial catabolism of long chain fatty acids to produce energy. FAO is a cyclic process that results in the shortening of fatty acids ((3-oxidation) and that generate in each cycle NADH, FADH₂, and acetyl-CoA. The generated acetyl-CoA enters the mitochondrial TCA cycle, where it is further oxidized to generate NADH and FADH₂. Finally, the NADH and FADH₂ produced by both beta-oxidation and the TCA cycle are used by the mitochondrial respiratory chain to produce ATP.

FAO is the major source of energy for skeletal muscle, heart and kidneys, while the liver oxidizes fatty acids primarily under the conditions of prolonged fasting, during illness, and during periods of increased physical activity. FAO also plays an essential role in the intermediary metabolism of the liver. Hepatic FAO fuels gluconeogenesis and the synthesis of ketone bodies, 3-hydroxy butyrate and acetoacetate, which are utilized as alternative sources of energy by extrahepatic organs, like the brain when blood glucose levels are low.

In addition to energy production, FAO is involved in regulation of cytosolic NADPH, the reducing agent that is key to support biosynthesis and redox homeostasis. Furthermore, several recent studies have identified the FAO-generated acetyl-CoA to be a carbon source for incorporation into aspartate (a nucleotide precursor), uridine monophosphate (a precursor of pyrimidine nucleoside triphosphates) and subsequently cellular DNA in endothelial cells.

Fatty acids are a family of molecules classified within the lipid macronutrient class. One role of fatty acids in animal metabolism is energy production, captured in the form of adenosine triphosphate (ATP). When compared to other macronutrient classes (carbohydrates and protein), fatty acids yield the most ATP on an energy per gram basis, when they are completely oxidized to CO2 and water by beta-oxidation and the TCA. Fatty acids (mainly in the form of triglycerides) are therefore the foremost storage form of fuel in most animals, and to a lesser extent in plants. In addition, fatty acids are important components of the phospholipids that form the phospholipid bilayers out of which all the membranes of the cell are constructed (the plasma membrane and other membranes that enclose all the organelles within the cells, such as the nucleus, the mitochondria, endoplasmic reticulum, and the Golgi apparatus). Fatty acids can also be cleaved, or partially cleaved, from their chemical attachments in the cell membrane to form second messengers within the cell, and local hormones in the immediate vicinity of the cell. The prostaglandins made from arachidonic acid stored in the cell membrane are probably the most well-known group of these local hormones.

Key enzymes in the FAO pathway are those belonging to the CPT system: Carnitine PalmitoylTransferase 1 (CPT1), Carnitine-acylcarnitine translocase (CACT), and Carnitine PalmitoylTransferase 2 (CPT2), as well as the 3-ketoacyl-coenzyme A thiolase (3-KAT).

According to the invention, the term “proteins according to the invention” or “proteins of the invention” will regroup the proteins (enzymes) of the CPT system and/or 3-KAT.

Thus the invention also relates to an inhibitor of CPT1 for use in the treatment of pancreatic cancer in a patient in need thereof.

Thus the invention also relates to an inhibitor of CACT for use in the treatment of pancreatic cancer in a patient in need thereof.

Thus the invention also relates to an inhibitor of CPT2 for use in the treatment of pancreatic cancer in a patient in need thereof.

Thus the invention also relates to an inhibitor of 3-KAT for use in the treatment of pancreatic cancer in a patient in need thereof.

As used herein, the term “CPT1” for “Carnitine PalmitoylTransferase 1” has its general meaning in the art and denotes the rate-limiting enzyme in FAO. CPT1 belongs to the CPT system, which is a multiprotein complex with catalytic activity within a core represented by CPT1 and CPT2 in the outer and inner mitochondrial membrane, respectively. CPT1 has two well-known isoforms, CPT1a and CPT1b, that are widely distributed in the human body and demonstrate considerable similarities. CPT1c is a new described isoform, but is primarily expressed in brain regions. CPT1 is responsible for importation of long-chain fatty acids into the mitochondria. In the cytosol, long-chain fatty acids are activated for degradation by conjugation with coenzyme A (CoA) to form long-chain fatty acyl-CoA. However, acyl-CoA molecules are unable to cross the mitochondrial membrane, thus CPT1 transfers the acyl groups from CoA to 1-carnitine resulting in the formation of acylcarnitines. The product is often Palmitoylcarnitine (thus the name) since Palmitoleic acid is one of the most abundant fatty acids of animal lipids, but other fatty acids may also be substrates. This “preparation” allows for subsequent movement of the acylcarnitine from the cytosol into the intermembrane space of mitochondria. Acylcarnitine and free carnitine are subsequently transported across the inner mitochondrial membrane by the carnitine translocase (CACT). CPT2 then transfer the acyl group back to CoA on the matrix side of the inner membrane before beta-oxidation.

CPT1a: UniProt accession number for the protein is P50416. Ensembl accession number for the gene is ENSG00000110090.

CPT1b: UnitProt accession number for the protein is Q92523. Ensembl accession number for the gene is ENSG00000205560.

CPT1c: UnitProt accession number for the protein is Q8TCG5. Ensembl accession number for the gene is ENSG00000169169.

Etomoxir, Perhexiline, Oxfenicine, ST1326 and Avocatin B are CPT-1 inhibitors (see for example the reference 37 or Yibao Maa et al., 2018).

As used herein, the term “CACT” for “Carnitine-acylcarnitine translocase” has its general meaning in the art and denotes a member of the mitochondrial carrier family of proteins (SLC25A20) that transport acylcarnitines across the inner mitochondrial membrane.

CACT: UniProt accession number for the protein is 043772. Ensembl accession number for the gene is ENSG00000178537.

S-nitrosoglutathione (GSNO) and Aminocarnitine are CACT inhibitors (see Tonazzi et al., 2017, and Chegary et al., 2008, respectively).

As used herein, the term “CPT2” for “Carnitine PalmitoylTransferase 2” has its general meaning in the art and denotes a peripheral inner-mitochondrial-membrane protein that reconvert the acylcarnitines generated by CPT1 into acyl-CoAs, before beta-oxidation.

Defects in this gene are associated with mitochondrial long-chain fatty acid (LCFA) oxidation disorders and CPT2 deficiency.

CPT2: UniProt accession number for the protein is P23786. Ensembl accession number for the gene is ENSG00000157184.

Aminocarnitine and Perhexiline are CPT2 inhibitors (see Yibao Maa et al., 2018).

As used herein, the term “3-KAT” for “3-ketoacyl-coenzyme A thiolase” (also called 0-ketothiolase, thiolase I or Acetyl-CoA acyltransferase) has its general meaning in the art and denotes the last enzyme that catalyzes the beta-oxidation of fatty acids. The 3-KAT enzyme belongs to the thiolases family and to the Mitochondrial Trifunctional Protein (MTP). The beta-oxidation is a cyclic process resulting in the progressive shortening (two carbons per cycle) of the fatty acids. Each cycle consist of four enzymatic steps, in which acylCoAs are dehydrogenated, hydrated and again dehydrogenated, and 3-KAT works in the last step to form acetyl-CoA and a new acyl-CoA, which is two carbons shorter than in the previous cycle. Acetyl-CoA fuels the TCA cycle, where it is further oxidized to generate NADH and FADH₂. In addition, besides acetyl-CoA, each β-oxidation-cycle generates NADH and FADH₂. Finally, the NADH and FADH₂ produced by both beta-oxidation and the TCA cycle are used by the mitochondrial respiratory chain to produce ATP.

3-KAT: UnitProt accession number for the protein is P42765. Ensembl accession number for the gene is ENSG00000167315.

Trimetazidine is an inhibitor of 3-KAT and Ranolazine is an anti-anginal drug with a clinical pharmacology similar to Trimetazidine (see for example the reference 37 or Yibao Maa et al., 2018).

More, the inventors show that an inhibitor of the fatty acid oxidation could be very suitable to sensitize pancreatic cancer cells to chemotherapeutic compounds already used to treat pancreatic cancer.

The therapeutic compounds used to treat pancreatic cancer refers to immune checkpoint inhibitor or chemotherapeutic compounds. Radiation therapy can be also used to treat pancreatic cancer.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

As used herein, the term “chemotherapeutic compounds” refers to chemical compounds that are effective in inhibiting tumor growth. Examples of chemotherapeutic agents include multkinase inhibitors such as sorafenib and sunitinib, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

These therapeutic compounds already used to treat pancreatic cancer are for example the gemcitabine, 5-fluorouracil (5-FU), Capecitabine, oxaliplatine, cisplatine, the irinotecan, FOLFIRINOX (a chemotherapy regimen made up of the following four drugs: FOL for folinic acid, F for fluorouracil (5-FU), IRIN for irinotecan and OX for oxaliplatin) or Nab-Paclitaxel.

Thus the invention also relates to an inhibitor of the fatty acid oxidation to sensitize pancreatic cancer cells to therapeutic compounds used to treat pancreatic cancer.

In another particular embodiment, the invention relates to an i) inhibitor of the fatty acid oxidation and a ii) therapeutic compound used to treat pancreatic cancer according to the invention as a combined preparation for simultaneous, separate or sequential use in the treatment of pancreatic cancer in a patient in need thereof or use in the sensitization of pancreatic cancerous cells in a patient in need thereof. Particularly, the therapeutic compound used to treat pancreatic can be the gemcitabine, the 5-fluorouracil (5-FU), the Capecitabine, the oxaliplatine, the cisplatine, the irinotecan, FOLFIRINOX (a chemotherapy regimen made up of the following four drugs: FOL for folinic acid, F for fluorouracil (5-FU), IRIN for irinotecan and OX for oxaliplatin) or Nab-Paclitaxel. In another particular embodiment, the invention relates to an i) inhibitor of CPT1 and a ii) therapeutic compound used to treat pancreatic cancer according to the invention as a combined preparation for simultaneous, separate or sequential use in the treatment of pancreatic cancer in a patient in need thereof or use in the sensitization of pancreatic cancerous cells in a patient in need thereof. Particularly, the compound is gemcitabine, the 5-fluorouracil (5-FU), the Capecitabine, the oxaliplatine, the cisplatine, the irinotecan, FOLFIRINOX (a chemotherapy regimen made up of the following four drugs: FOL for folinic acid, F for fluorouracil (5-FU), IRIN for irinotecan and OX for oxaliplatin) or Nab-Paclitaxel.

In another particular embodiment, the invention relates to an i) inhibitor of CPT2 and a ii) therapeutic compound used to treat pancreatic cancer according to the invention as a combined preparation for simultaneous, separate or sequential use in the treatment of pancreatic cancer in a patient in need thereof or use in the sensitization of pancreatic cancerous cells in a patient in need thereof. Particularly, the compound is gemcitabine, the 5-fluorouracil (5-FU), the Capecitabine, the oxaliplatine, the cisplatine, the irinotecan, FOLFIRINOX (a chemotherapy regimen made up of the following four drugs: FOL for folinic acid, F for fluorouracil (5-FU), IRIN for irinotecan and OX for oxaliplatin) or Nab-Paclitaxel.

In another particular embodiment, the invention relates to an i) inhibitor of 3-KAT and a ii) therapeutic compound used to treat pancreatic cancer according to the invention as a combined preparation for simultaneous, separate or sequential use in the treatment of pancreatic cancer in a patient in need thereof or use in the sensitization of pancreatic cancerous cells in a patient in need thereof. Particularly, the compound is gemcitabine, the 5-fluorouracil (5-FU), the Capecitabine, the oxaliplatine, the cisplatine, the irinotecan, FOLFIRINOX (a chemotherapy regimen made up of the following four drugs: FOL for folinic acid, F for fluorouracil (5-FU), IRIN for irinotecan and OX for oxaliplatin) or Nab-Paclitaxel.

In another particular embodiment, the invention relates to an i) inhibitor of the fatty acid oxidation and a ii) radiation therapy as a combined preparation for simultaneous, separate or sequential use in the treatment of pancreatic cancer in a patient in need thereof or use in the sensitization of pancreatic cancerous cells in a patient in need thereof.

As used herein, the term “radiation therapy” has its general meaning in the art and refers the treatment of pancreatic cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

According to the invention, the pancreatic cancer can be a Pancreatic Ductal AdenoCarcinoma (PDAC).

As used herein, the term “Pancreatic Ductal Aden® Carcinoma” or “PDAC” has its general meaning in the art and refers to pancreatic ductal adenocarcinoma such as revised in the World Health Organization Classification C25.

As used herein, the term “patient” denotes a mammal, such as a rodent, a feline, a canine, and a primate. Particularly, the patient according to the invention is a human. More particularly, the patient is a human suffering of a pancreatic cancer.

As used herein, the term “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The term “inhibitor of fatty acid oxidation” denotes molecules or compounds which can inhibit the catabolic process by which mitochondria break down fatty acids into acetyl-CoA to generate energy. Particularly, FAO inhibitors are pharmacological compounds that target CPT1 or 3-KAT (last step of beta-oxidation).

The term “inhibitor of CPT1” denotes molecules or compounds which can inhibit or destabilize the activity/function of the enzyme and particularly the formation of acyl carnitines. According to the invention, “inhibitor of CPT1” denotes an inhibitor of CPT1a or CPT1b, more particularly an inhibitor of CPT1a.

The term “inhibitor of CACT” denotes molecules or compounds which can inhibit or destabilize the activity/function of the mitochondrial carrier protein and particularly the transport of acylcarnitines across the inner mitochondrial membrane.

The term “inhibitor of CPT2” denotes molecules or compounds which can inhibit or destabilize the activity/function of the enzyme and particularly the formation of acyl-CoAs before enter beta oxidation in the mitochondria.

The term “inhibitor of 3-KAT” denotes molecules or compounds which can inhibit or destabilize the activity/function of the enzyme and particularly the fatty acid beta-oxidation.

The inventors also showed that the use of an inhibitor of the FAO or the combination of an inhibitor of FAO with a chemotherapeutic compound used to treat pancreatic cancer (as described above) result in a complete tumor regression in High OXPHOS PDAC xenograft. Thus, this kind of treatments could be used in patient suffering of a pancreatic cancer with a high OXPHOS status.

Thus, the invention also relates to an inhibitor of fatty acid oxidation (FAO) for use in the treatment of pancreatic cancer in a patient with a high OXPHOS profile (high OXPHOS status).

Particularly, the invention also relates to an i) inhibitor of the fatty acid oxidation and a ii) therapeutic compound used to treat pancreatic cancer according to the invention as a combined preparation for simultaneous, separate or sequential for use in the treatment of pancreatic cancer in a patient in need thereof or for use in the sensitization of pancreatic cancerous cells in patients with high OXPHOS profile (high OXPHOS status).

As used herein, the term “OXPHOS” denotes the metabolic pathway in which ATP is formed as a result of the transfer of electrons from NADH or FADH₂ to 02 by a series of electron carriers. This process, which takes place in mitochondria, is the major source of ATP in aerobic organisms. Determining a high or low OXPHOS status is well known in the art. For example and according to the invention, the measurement of the “level of OXPHOS” (or “OXPHOS status”) can be done by measurement of the cellular oxygen consumption rate (OCR). This kind of measurement can be easily done directly on a sample, for example pancreatic cancerous cells obtained from a patient, thanks to a device like Seahorse (see for example: https://www.agilent.com/en/products/cell-analysis/seahorse-analyzers). Then, determining a high or a low “level of OXPHOS” will be easily obtained by comparing the obtained level to threshold (reference value) obtained with control (like with patients with no pancreatic cancer for example).

The inventors determined a correlation between response to FAO inhibitor and the OXPHOS status (see Table 1). Namely, high and intermediate responder to FAO inhibitor, such as Perhexiline, correspond to patients with high OXPHOS profile (high OXPHOS status). Low responder to FAO inhibitor corresponds to patients with low OXPHOS profile (low OXPHOS status).

The inventors also determined a correlation between the expression levels of genes of the different Complex (complexes I to V) of the mitochondria with the OXPHOS status. A high expression of these genes is correlated with a high OXPHOS.

Thus, the expression level of at least one gene expressed in the mitochondrial respiratory Complexes (Complexes I to V) of the mitochondria can be measured to determine the OXPHOS status.

The genes for the Complex I are selected in the group consisting in ND1, ND2, ND3, ND4, ND4L, ND5, ND6, NDUFB2, NDUFAF4, NDUFAF6, NDUFAF2, NDUFA12, NDAUFAF3, NDUFAF5, NDUFA5, NDUFB6, NDUFS4, NDUFA13, NDUFV2, NUBPL, NDUFB4, NDUFS7, NDUFB8, NDUFAF7, NDUFB3, NDUFS5, NDUFA11, NDUFC1, NDUFB10, NDUFS8, NDUFA10, NDUFB9, NDUFA7, NDUFA1, NDUFB5, NDUFB7, NDUFA2, NDUFB1, TMEM126B, NDUFB11, NDUFS6, NDUFV3, NDUFS3, NDUFS2, NDUFA8, NDUFV1, NDUFAB1, PARK7/DJ1, ND6, NDUFA6, NDUFS1, NDUFA9, TIMMDC1.

The genes for the Complex II are selected in the group consisting in SDHAF1, SDHB, SDHAF2, SDHAF3, SDHD, SDHC, SDHA.

The genes for the Complex III are selected in the group consisting in cytochrome b, LYRM7, UQCRB, UQCRQ, BCS1L, UQCR11, TTC19, UQCRFS1, UQCRC1, UQCR10, CYC1, UQCRC2, AARS2, UQCC3, UQCC2, UQCC1, TTC19, BCS1L, MAIP1, IMMP1L, SAMM50, IMMP2L, SLC25A33.

The genes for the Complex IV are selected in the group consisting in COX1, COX2, COX3, COX4, COX5A, COX5B, COX6A, COX6B, COX6C, COX7A, COX7B, COX7C, COX8, PET100, TIMM21, SELRC1, NDUFA4, COX20, SURF1, CAV3, COX18, ANK3, SERP1, SCO1, COX15, MT-CO3, A4GALT, SPTBN1, COX11, COX10, COA6.

The genes for the Complex V are selected in the group consisting in A6, A8, ATP5F1A, ATP5A, ATP5F1B, ATP5F1C, ATP5F1D, ATP5F1E, ATP5MC1, ATP5MC2, ATP5MC3, ATP5MD, ATP5ME, ATP5MF, ATP5MG, ATP5MPL, MT-ATP6, MT-ATP8, ATP5PB, ATP5PD, ATP5PF, ATP5PO, ATP5IF1.

More particularly, the expression level of one gene by Complexes (thus a total of five genes) can be measured.

More particularly, the expression level of at least one gene selected in the group consisting in ATP5A, UQCRC2, SDHB, COX2 and NDUFB8 can be used to determine the level of OXPHOS.

More particularly, the expression level of the gene NDUFB8 can be used to evaluate the level of activity of the Complex I and thus the level of OXPHOS.

More particularly, the expression level of the gene SDHB can be used to evaluate the level of activity of the Complex II and thus the level of OXPHOS.

More particularly, the expression level of the gene UQCRC2 can be used to evaluate the level of activity of the Complex III and thus the level of OXPHOS.

More particularly, the expression level of the gene COX2 can be used to evaluate the level of activity of the Complex IV and thus the level of OXPHOS.

More particularly, the expression level of the gene ATP5A can be used to evaluate the level of activity of the Complex V and thus the level of OXPHOS.

As used herein, the term “NDUFB8” for “NADH dehydrogenase [ubiquinone] 1 beta subcomplex subunit 8, mitochondrial” denotes an enzyme that in humans is encoded by the NDUFB8 gene. NADH dehydrogenase (ubiquinone) 1 beta subcomplex subunit 8 is an accessory subunit of the NADH dehydrogenase (ubiquinone) Complex, located in the mitochondrial inner membrane. It is also known as Complex I and is the largest of the five Complexes of the electron transport chain. The Entrez reference number is 4714.

As used herein, the term “SDHB” for “Succinate dehydrogenase [ubiquinone] iron-sulfur subunit, mitochondrial” also known as iron-sulfur subunit of Complex II (Ip) denotes a protein that in humans is encoded by the SDHB gene. The succinate dehydrogenase (also called SDH or Complex II) protein Complex catalyzes the oxidation of succinate (succinate+ubiquinone=>fumarate+ubiquinol). SDHB is one of four protein subunits forming succinate dehydrogenase, the other three being SDHA, SDHC and SDHD. The SDHB subunit is connected to the SDHA subunit on the hydrophilic, catalytic end of the SDH Complex. It is also connected to the SDHC/SDHD subunits on the hydrophobic end of the Complex anchored in the mitochondrial membrane. The Entrez reference number is 6390.

As used herein, the term “UQCRC2” for “Cytochrome b-c1 Complex subunit 2, mitochondrial” also known as QCR2, UQCR2, or MC3DN5 denotes a protein that in humans is encoded by the UQCRC2 gene. The product of UQCRC2 is a subunit of the respiratory chain protein Ubiquinol Cytochrome c Reductase (UQCR, Complex III or Cytochrome bc1 Complex), which consists of the products of one mitochondrially encoded gene, MTCYTB (mitochondrial cytochrome b) and ten nuclear genes: UQCRC1, UQCRC2, Cytochrome c1, UQCRFS1 (Rieske protein), UQCRB, “11 kDa protein”, UQCRH (cyt c1 Hinge protein), Rieske Protein presequence, “cyt. c1 associated protein”, and “Rieske-associated protein.” Defects in UQCRC2 are associated with mitochondrial Complex III deficiency, nuclear, type 5. The Entrez reference number is 7385.

As used herein, the term “COX2” for “cytochrome c oxidase subunit 2” also known as cytochrome c oxidase polypeptide II denotes a protein that in humans is encoded by the MT-CO2 gene.[4] Cytochrome c oxidase subunit II, abbreviated COX2, COXII, COII, or MT-CO2, is the second subunit of cytochrome c oxidase (Complex IV). The Entrez reference number is 4513.

As used herein, the term “ATP5A” for “synthase-coupling factor 6, mitochondrial” denotes an enzyme that in humans is encoded by the ATP5PF gene. It's a subunit of mitochondrial ATP synthase (Complex V). The Entrez reference number is 522.

In a particular embodiment the OXPHOS level of a patient can be determined by determining by measurement of the cellular oxygen consumption rate (OCR) and/or of the expression level of at least one gene expressed in the mitochondrial respiratory Complexes (Complexes I to V) of the mitochondria and especially the genes ATP5A, UQCRC2, SDHB, COX2 and NDUFB8. In a particular embodiment the level of OXPHOS is determined by both methods.

Measuring the expression level of the genes listed above can be done by measuring the gene expression level of these genes or by measuring the level of the protein of the corresponding genes and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Methods of the invention may comprise a step consisting of comparing the genes expression (by the mRNA) with a “control value” or “cut-off” or “threshold”.

Predetermined reference values used for comparison may comprise “cut-off” or “threshold” values that may be determined as described herein. Each reference (“cut-off”) value for the genes' expression may be predetermined by carrying out a method comprising the steps of

a) providing a collection of samples from patients suffering a pancreatic cancer (after diagnosis of the cancer for example);

b) determining the expression level of the genes for each sample contained in the collection provided at step a);

c) ranking the tumor tissue samples according to said gene expression level and determining a threshold value above which the expression level is said to be “high” and below which the expression level is said to be “low”;

d) quantitatively defining the threshold/cut-off/reference value by determining the number of copies of the said gene corresponding to the threshold/cut-off/reference value; to be done by constructing a calibration curve using known input quantities of cDNA or protein for the said gene/protein;

e) classifying said samples in pairs of subsets of increasing, respectively decreasing, number of members ranked according to their expression level,

f) providing, for each sample provided at step a), information relating to the actual OXPHOS status for the corresponding cancer patient (i.e. the duration of the overall survival (OS));

g) for each pair of subsets of samples, obtaining a Kaplan Meier percentage of survival curve;

h) for each pair of subsets of samples calculating the statistical significance (p value) between both subsets

i) selecting as reference value for the expression level, the value of expression level for which the p value is the smallest.

In one embodiment, the inhibitors according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

Compounds useful for inhibiting FAO are well known in the art (see for example the references 37 to 41 and Yibao Maa et al., 2018).

Thus, inhibitors of the FAO can be inhibitors of the enzyme CPT1 like Etomoxir, Perhexiline, Oxfenicine, Methyl palmoxirate, S-15176, Metoprolol, and amiodarone, can be inhibitors of CPT2 like Aminocarnitine and Perhexiline or inhibitors of CACT like S-nitrosoglutathione (GSNO) or Aminocarnitine Acid (see Tonazzi et al., 2017, and Chegary et al., 2008).

Inhibitors of FAO can also be the mildronate (carnitine biosynthesis inhibitor, see for example Liepinsh, E yet al., 2006), Trimetazidine, Ranolazine and 4-bromocrotonic acid (thiolase inhibitors) or the pFOX which directly inhibits the fatty acid beta-oxidation.

According to the invention, the term “inhibitor according to the invention” will regroup inhibitor of FAO, inhibitor of CPT1, CACT, CPT2 and/or inhibitor of 3-KAT.

In a particular embodiment, the inhibitor according to the invention is an antibody. Antibodies directed against the proteins (enzyme) of the invention (like the proteins CPT1, CACT, CPT2 and 3-KAT) can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against the proteins of the invention (like the proteins CPT1, CACT, CPT2 and 3-KAT) can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce single chain antibodies. Compounds useful in practicing the present invention also include antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to the proteins of the invention (like the proteins CPT1, CACT, CPT2 and 3-KAT).

Humanized antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies are selected.

In another embodiment, the antibody according to the invention is a single domain antibody against the proteins of the invention (like the proteins CPT1, CACT, CPT2 and 3-KAT). The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers are selected.

In one embodiment, the compound according to the invention is a polypeptide.

In a particular embodiment the polypeptide is an antagonist of the proteins of the invention (like the proteins CPT1, CACT, CPT2 and 3-KAT) and is capable to prevent their functions.

In one embodiment, the polypeptide of the invention may be linked to a cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group Complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the inhibitor according to the invention is an inhibitor of the gene expression of the protein of the invention (like the genes expressing for the protein CPT1, CACT, CPT2 and 3-KAT here the “genes CPT1, CACT, CPT2 and 3-KAT”).

Small inhibitory RNAs (siRNAs) can also function as inhibitors of the genes expression of the invention (like the genes CPT1, CACT, CPT2 and 3-KAT) for use in the present invention. Genes expression of the invention (like the genes CPT1, CACT, CPT2 and 3-KAT) can be reduced by contacting a patient or cell with a small double stranded RNA (dsRNA), or a vector or construct causing the production of a small double stranded RNA, such that the genes expression of the invention (like the genes CPT1, CACT, CPT2 and 3-KAT) are specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of the genes expression of the genes expressing the proteins of the invention (like the genes expressing for the proteins CPT1, CACT, CPT2 and 3-KAT) for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of the genes expression of the proteins of the invention (like the genes expressing for the proteins CPT1, CACT, CPT2, and 3-KAT) can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In a particular embodiment, an endonuclease can be used to reduce or abolish the expression of the genes expression of the invention (like the genes expressing for the proteins CPT1, CACT, CPT2 and 3-KAT).

Indeed, as an alternative to more conventional approaches, such as cDNA overexpression or downregulation by RNA interference, new technologies provide the means to manipulate the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone non homologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In order to test the functionality of a putative inhibitor of the mitochondrial respiration a test is necessary. For that purpose, to identify inhibitors of the mitochondrial respiration, we can follow mitochondrial respiration by measuring oxygen consumption rate with Seahorse after acute or chronic treatment of cells with the putative drug. An inhibition of the oxygen consumption will be the demonstration that the tested drug is an inhibitor of the mitochondrial respiration.

### Therapeutic Composition

Another object of the invention relates to a therapeutic composition comprising an inhibitor of fatty acid oxidation (FAO) according to the invention for use in the treatment of pancreatic cancer in a patient in need thereof.

In another particular embodiment, the invention relates to a therapeutic composition comprising an inhibitor of the mitochondrial respiration according to the invention for use in the treatment of a pancreatic cancer in a patient with a high OXPHOS profile (high OXPHOS status) as determined in the method above.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise at least one further therapeutic active agent. The present invention also relates to a kit comprising inhibitors according to the invention and at least one further therapeutic active agent.

For example, anti-cancer agents may be added to the pharmaceutical composition as described below.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and

Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum Complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

### Predicting and Monitoring Method

Another aspect of the invention relates to an in vitro method for predicting FAO inhibitor response of a patient in need thereof, comprising: i) determining, in a sample obtained from the patient, the expression level of the CPT1C isoform; ii) comparing the expression level of the CPT1C isoform determined at step i) with a reference values and iii) concluding that the patient will respond to FAO inhibitor response when the expression level determined at step i) is lower than the reference value.

In some embodiment, the FAO inhibitor is an inhibitor of CPT1.

In some embodiment, the CPT1 inhibitor is Etomoxir, Perhexiline, Oxfenicine, Methyl palmoxirate, S-15176, Metoprolol, or amiodarone.

In some embodiment, the CPT1 inhibitor is Perhexiline.

As used herein, the term “sample’ denotes blood, fresh whole blood, peripheral-blood, peripheral blood mononuclear cell (PBMC) and tumor sample.

In a preferred embodiment, the sample is tumor sample. As used herein, the term “tumor sample” means any tissue tumor sample derived from the subject. Said tissue sample is obtained for the purpose of the in vitro evaluation. In some embodiments, the tumor sample may result from the tumor resected from the subject. In some embodiments, the tumor sample may result from a biopsy performed in the primary tumor of the subject or performed in metastatic sample distant from the primary tumor of the subject. In some embodiments, the tumor sample is a sample of circulating tumor cells. As used herein, the term “circulating tumor cell” or “CTC” refers to a cancer cell derived from a cancerous tumor that has detached from the tumor and is circulating in the blood stream of the subject. Typically the CTCs are isolated from the blood sample using a filter and/or a marker based method. In some embodiments, the tumor sample is a sample of PDAC primary cells.

As used herein, a “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the gene expression level (obtained according to the method of the invention) with a defined threshold value

Each reference (“cut-off”) value for the genes' expression may be predetermined by carrying out a method comprising the steps of

a) providing a collection of samples from patients suffering of pancreatic cancer (after diagnosis of pancreatic cancer for example);

b) determining the expression level of the CPT1C isoform for each sample contained in the collection provided at step a)

c) ranking the samples according to CPT1C isoform expression level determined and determining a threshold value above which the expression level is said to be “high” and below which the expression level is said to be “low”;

d) quantitatively defining the threshold/cut-off/reference value by determining the number of copies of the said gene corresponding to the threshold/cut-off/reference value; to be done by constructing a calibration curve using known input quantities of cDNA or protein for the said gene;

e) classifying said samples in pairs of subsets of increasing, respectively decreasing, number of members ranked according to their expression level,

f) providing, for each sample provided at step a), information relating to the actual treatment outcome for the corresponding patient (i.e. good or poor responders after treatment with FAO inhibitor);

g) for each pair of subsets of samples, obtaining a cluster of expression fold changes using Euclidian distance.

h) for each pair of subsets of samples calculating the statistical significance (p value) between both subsets

i) selecting as reference value for the expression level, the value of expression level for which the p value is the smallest.

For example the expression level of the genes has been assessed for 100 samples from 100 patients. The 100 samples are ranked according to their expression level. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual treatment outcome for the corresponding patient, Euclidian distance are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels. In routine work, the reference value (cut-off value) may be used in the present method to discriminate good and poor antipsychotic responders.

Euclidian distances are commonly used to measure the dissimilarity between expression profiles with regard to the signature genes and are well known by the person skilled in the art. The man skilled in the art also understands that the same technique of assessment of the expression level of a gene should of course be used for obtaining the reference value and thereafter for assessment of the expression level of a gene of a patient subjected to the method of the invention.

Measuring the expression level of the CPT1C isoform listed above can be done by measuring the gene expression level of CPT1C isoform and can be performed by a variety of techniques well known in the art.

Typically, the expression level of a gene may be determined by determining the quantity of mRNA. Methods for determining the quantity of mRNA are well known in the art. For example, the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence-based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfor1ic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as 3 H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pir1kel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In another embodiment, the expression level is determined by metabolic imaging (see for example Yamashita T et al., Hepatology 2014, 60:1674-1685 or Ueno A et al., Journal of hepatology 2014, 61:1080-1087).

Expression level of a gene may be expressed as absolute expression level or normalized expression level. Typically, expression levels are normalized by correcting the absolute expression level of a gene by comparing its expression to the expression of a gene that is not a relevant for determining the response of FAO inhibitor treatment, e.g., a housekeeping gene that is constitutively expressed.

According to the invention, the expression level of CPT1C isoform may also be measured by measuring the protein expression level encoding by said gene and can be performed by a variety of techniques well known in the art.

Typically protein expression level may be measured for example by capillary electrophoresis-mass spectroscopy technique (CE-MS), flow cytometry, mass cytometry or ELISA performed on the sample.

In the present application, the “level of CPT1C isoform” or the “CPT1C isoform level expression” means the quantity or concentration of said CPT1C isoform. In one embodiment, the “level of CPT1C” means the quantitative measurement of the CPT1C isoform expression relative to a negative control.

Such methods comprise contacting a sample with a binding partner capable of selectively interacting with CPT1C isoform present in the sample. The binding partner is generally an antibody that may be polyclonal or monoclonal, preferably monoclonal.

The presence of the CPT1C isoform can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS). etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labelled secondary binding molecule is added. The secondary binding molecule is allowed to react with any captured sample marker protein, the plate is washed and the presence of the secondary binding molecule is detected using methods well known in the art.

Particularly, a mass spectrometry-based quantification methods may be used. Mass spectrometry-based quantification methods may be performed using either labelled or unlabelled approaches [DeSouza and Siu, 2012]. Mass spectrometry-based quantification methods may be performed using chemical labelling, metabolic labelling or proteolytic labelling. Mass spectrometry-based quantification methods may be performed using mass spectrometry label free quantification, a quantification based on extracted ion chromatogram (EIC) and then profile alignment to determine differential level of polypeptides.

Particularly, a mass spectrometry-based quantification method particularly useful can be the use of targeted mass spectrometry methods as selected reaction monitoring (SRM), multiple reaction monitoring (MRM), parallel reaction monitoring (PRM), data independent acquisition (DIA) and sequential window acquisition of all theoretical mass spectra (SWATH) [Moving target Zeliadt N 2014 The Scientist; Liebler Zimmerman Biochemistry 2013 targeted quantitation pf proteins by mass spectrometry; Gallien Domon 2015 Detection and quantification of proteins in clinical samples using high resolution mass spectrometry. Methods v81 p 15-23; Sajic, Liu, Aebersold, 2015 Using data-independent, high-resolution mass spectrometry in protein biomarker research: perspectives and clinical applications. Proteomics Clin Appl v9 p 307-21].

Particularly, the mass spectrometry-based quantification method can be the mass cytometry also known as cytometry by time of flight (CYTOF) (Bandura D R, Analytical chemistry, 2009).

Particularly, the mass spectrometry-based quantification is used to do peptide and/or protein profiling can be use with matrix-assisted laser desorption/ionisation time of flight (MALDI-TOF), surface-enhanced laser desorption/ionization time of flight (SELDI-TOF; CLINPROT) and MALDI Biotyper apparatus [Solassol, Jacot, Lhermitte, Boulle, Maudelonde, Mange 2006 Clinical proteomics and mass spectrometry profiling for cancer detection. Journal: Expert Review of Proteomics V3, 13, p 311-320; FDA K130831].

In another aspect, the invention relates to an in vitro method for monitoring FAO inhibitor treatment in a subject in need thereof, comprising the steps of i) determining in a sample obtained from said patients the expression level of CPT1C isoform; ii) comparing the expression level measured at step i) with a reference value, and iii) concluding that the FAO inhibitor treatment is efficient when the level determined at step ii) is lower than the reference value.

In some embodiment, the reference value is the expression level of CPT1C determined in samples obtained from the subject before FAO inhibitor treatment.

Thus, the invention relates to an in vitro method for monitoring FAO inhibitor treatment in a subject in need thereof, comprising the steps of i) determining in a sample obtained before FAO inhibitor treatment from said patients the expression level of CPT1C isoform; ii) determining in a sample obtained after FAO inhibitor treatment from said patients the expression level of CPT1C isoform iii) comparing the expression level measured at step i) with the expression level determined at step ii), and iii) concluding that the FAO inhibitor treatment is efficient when the level determined at step ii) is lower than the expression level determined at step i).

The patient is considered as good responder when it is concluded that the FAO inhibitor treatment is efficient.

The patient is considered as poor responder when it is concluded that the FAO inhibitor treatment is not efficient (i.e CPT1C expression level is higher than the reference value).

In another aspect, the invention relates to a method for monitoring FAO inhibitor treatment in patient suffering from pancreatic cancer comprising the steps of i) measuring in a sample obtained from said patients the expression level of CPT1C isoform; ii) comparing the expression level measured at step i) with a reference value, and iii) administering a therapeutically effective amount of FAO inhibitor when the gene level determined at step i) is lower than the reference value.

In other word, the invention relates to a method for treating pancreatic cancer in patient in need thereof comprising the step of i) determining if the patient will respond to FAO inhibitor according to the method of the invention and ii) administering to said patient a therapeutically effective amount of FAO inhibitor when the patient is determined as responder of FAO inhibitor.

The invention will be further illustrated by the following figures and examples. However, these examples and figures should not be interpreted in any way as limiting the scope of the present invention.

### FIGURES

FIG. 1. PDAC cells exhibit different sensitivity to pharmacological FAO inhibitors.

A. Relative cell viability of PDAC cells treated with Etomoxir (62.5 μM) for 72 h, compared to non-treated cells. The dotted line represents the mean of the treated cells (0.75). B. Relative cell viability of PDAC cells treated with Perhexiline (7 μM) for 72 h, compared to non-treated cells. The dotted line represents the mean of the treated cells (0.72).

C. Relative cell viability of PDAC cells treated with Trimetazidine (62.5 μM) for 72 h, compared to non-treated cells.

Data are presented as the mean of triplicates±SEM, and are representative of three independent experiments.

Each number below the bars corresponds to the number of the anonymized name of patients (for example 84 corresponds to patient PDAC084T as used in FIGS. 3 to 8). Viability is measured by Crystal violet assay.

FIG. 2. The OXPHOS rates correlate with sensitivity to FAO inhibition in PDAC cells.

Scatterplot of the basal Oxygen Consumption Rate (OCR) versus relative mean cell viability after 72 hours-treatment with Etomoxir (62.5 μM) or Perhexiline (7 μM) in PDAC cells shown in FIGS. 2A and 2B, respectively.

The OCR is significantly correlated with the relative cell viability for Perhexiline treatment, with a Pearson correlation coefficient (r) of −0.8186 (negative correlation).

No significant negative correlation is observed between Etomoxir treatment and relative cell viability (r=−0.5501).

Pearson correlation and two-tailed t-test were used to generate the correlation coefficient and associated P value.

FIG. 3. The FAO inhibitor Perhexiline in combination with Gemcitabine is synergistic specifically in high OXPHOS cells

Dose-response assays were performed in four selected PDAC cells: PDAC084T (FIG. 3A), PDAC012T (FIG. 3B), PDAC032T (FIG. 3C), and PDAC022T (FIG. 3D), according to the sensitivities to the FAO inhibitors. Cells were treated for 72 h with increasing doses of Gemcitabine, with Perhexiline (5 μM) or with the combination. Cell viability was measured by Crystal violet viability assay. Figures show the cell viability (%) of each treatment: Perhexiline (5 μM), Gemcitabine at one dose (1 nM), or the combination.

To determine the synergistic effect in the combination treatments, we calculated the predicted values by multiplying the cell viability in the Gemcitabine and Perhexiline groups. Then, when the observed value for the combination is less than the predicted value, we consider it as a synergistic effect. Data are presented as mean±SEM of triplicates. P values from Student's t-test.

FIG. 4. Impact of FAO inhibition in subcutaneous PDAC xenografts. Tumor growth progression in immunodeficient mice treated during one month with Gemcitabine (120 mg/kg IP twice a week), Perhexiline (5 mg/kg IP every other day), Gemcitabine plus Perhexiline (with the same indications), and vehicle.

A. Perhexiline not only enhances the antitumoral effect of Gemcitabine but also results in complete tumoral regression in a High OXPHOS PDAC xenograft (PDAC084T). B. The combination treatment shows a better efficiency than Gemcitabine alone in the intermediate responder xenograft (PDAC012T). C, D. No effect was seen upon combination treatment compared to Gemcitabine alone in the low responder xenografts (PDAC032T, PDAC022T). Data are presented as mean±SEM. ***P<0.001, **P<0.01, *P<0.05 from Two-way ANOVA test, compared to Gemcitabine treatment alone.

FIG. 5. The FAO inhibitor Perhexiline enhances the antitumor activity of chemotherapy by inducing apoptosis in vitro. Cell survival was measured using an AnnexinV/PI apoptosis assay and viable cells (AnnexinV⁻/PI⁻ cells) were quantified. A. Quantification showing the cell survival (%) of four cell lines treated with Perhexiline (7 μM) for 72 h compared with the controls. Data are presented as mean±SEM of three independent experiments performed in duplicates. B. Quantification showing the viability of PDAC084T cells treated for 24 h with Gemcitabine (1 μm) Perhexiline (7 μm) or the combination. Data are presented as mean±SEM of one experiment performed in duplicates. P values from Student's t-test.

FIG. 6. The FAO inhibitor Perhexiline enhances the antitumor activity of chemotherapy by inducing apoptosis in vivo. Quantification of cleaved-Caspase 3 (A), Ki67 (B), and Masson's Trichrome staining (C), in tumor sections from PDAC084T xenografts treated with Gemcitabine, or the combination Perhexiline plus Gemcitabine.

FIG. 7. The FAO inhibitor Perhexiline enhances the antitumor activity of chemotherapy by inducing energetic stress in vitro. A. Mitochondrial respiration (Oxygen Consumption Rate, OCR) was measured on Seahorse oxygraph in PDAC084T cells after 6 h of Perhexiline, Gemcitabine, or combination treatment. B. The basal and maximal respiration, ATP production by mitochondria, and spare respiratory capacity were calculated.

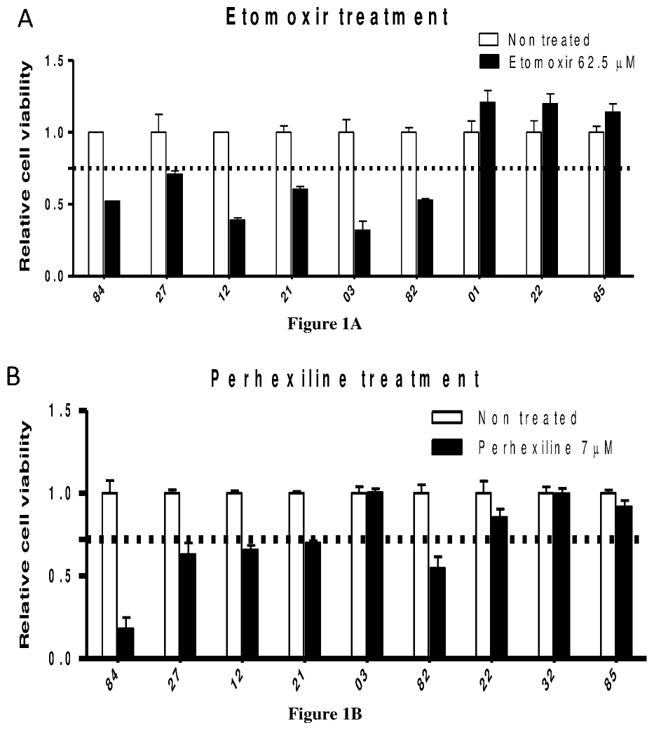

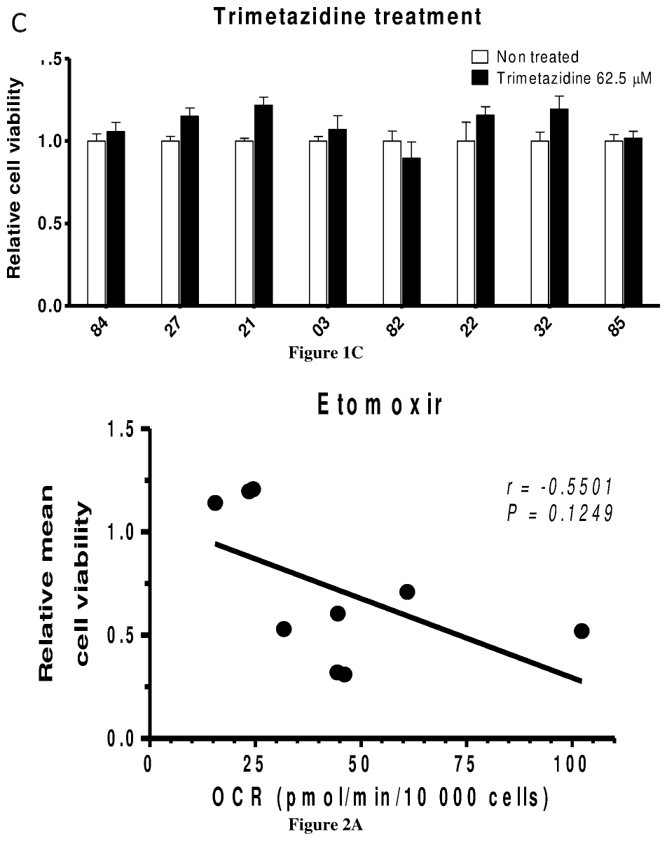

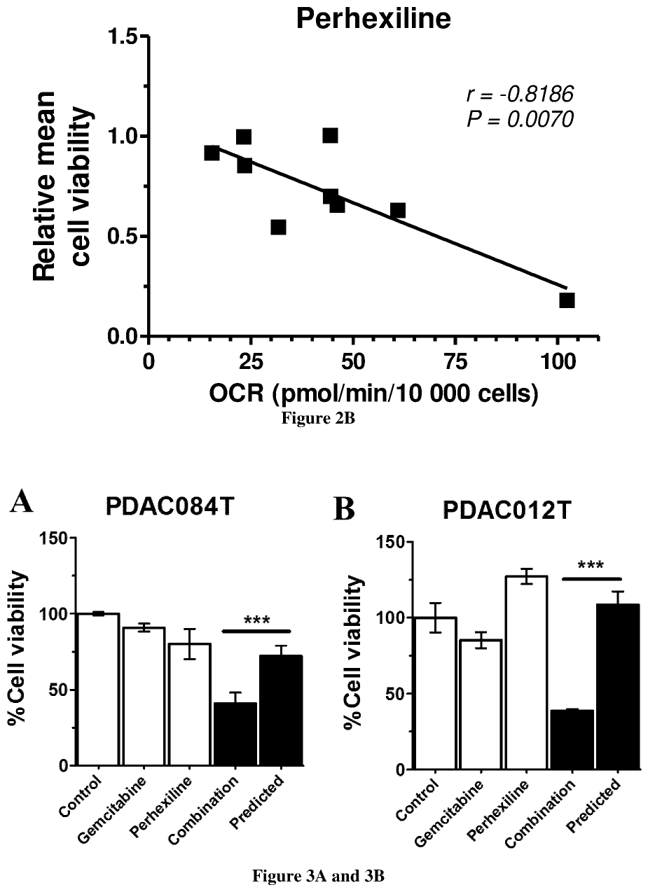

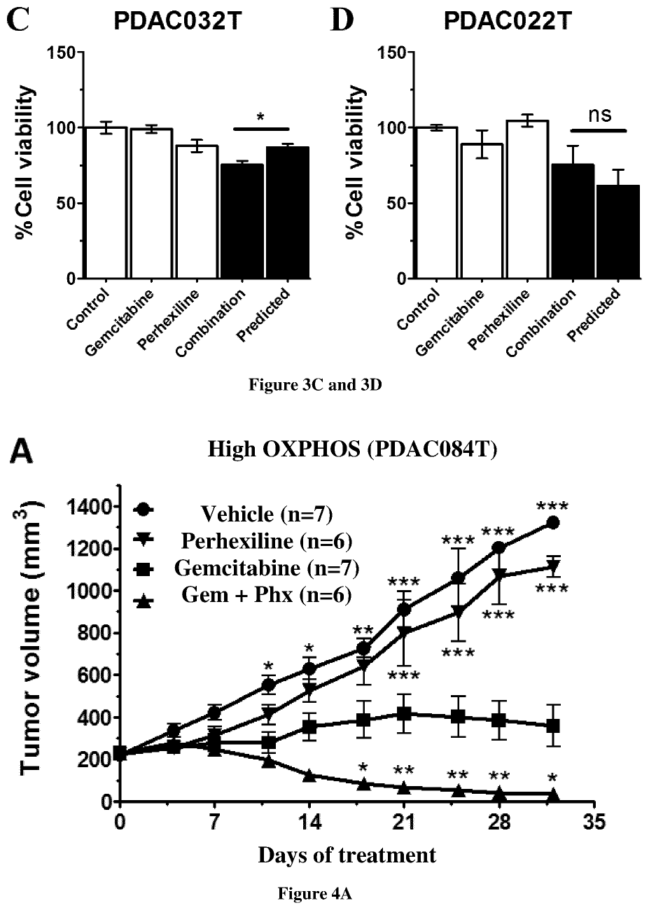

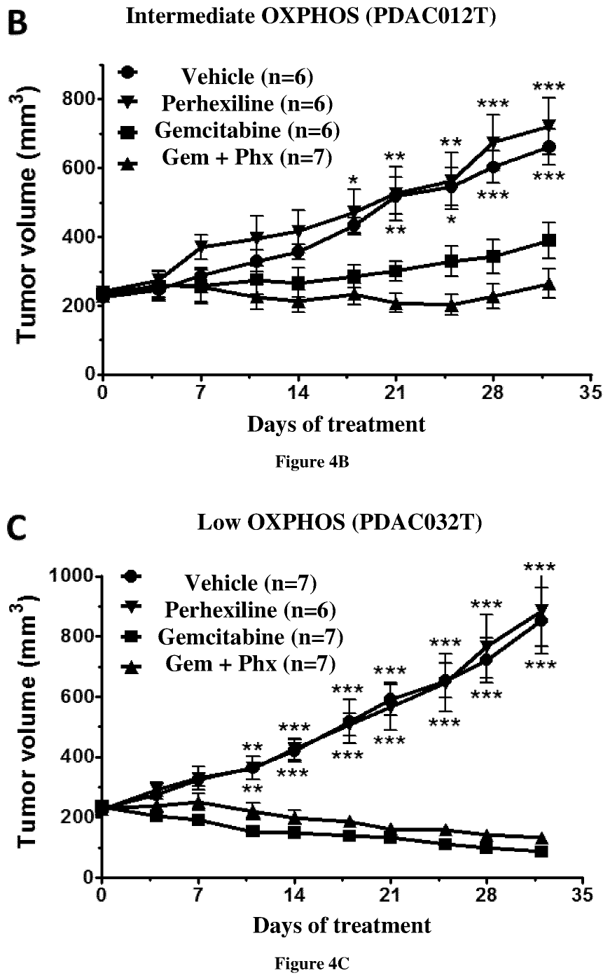

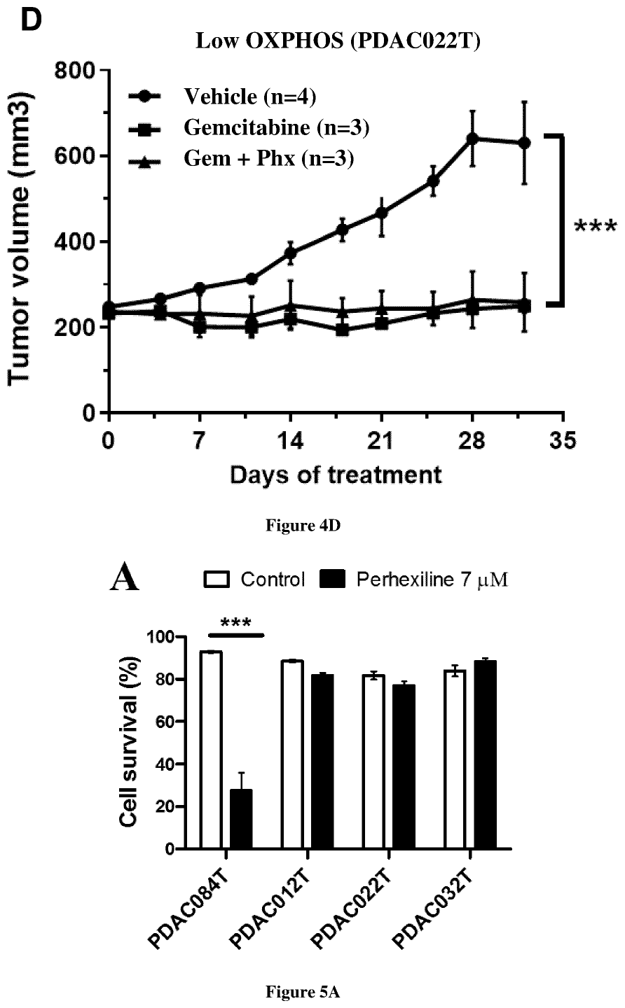

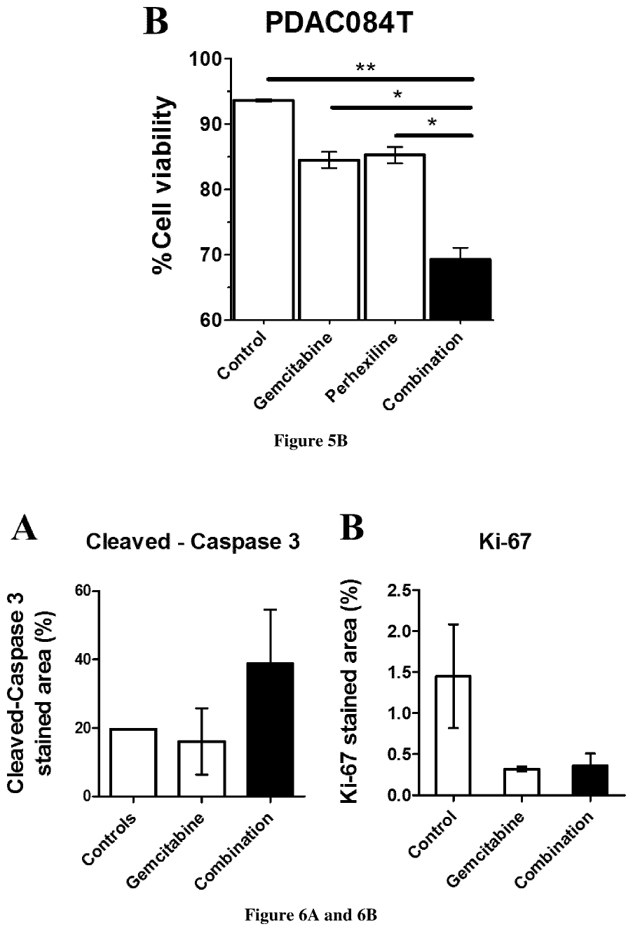

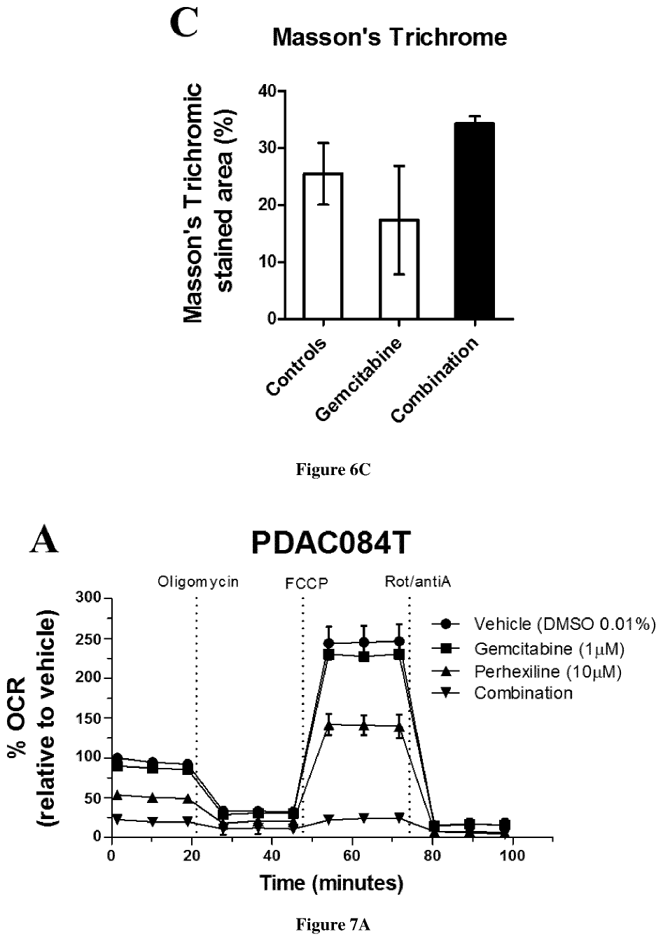

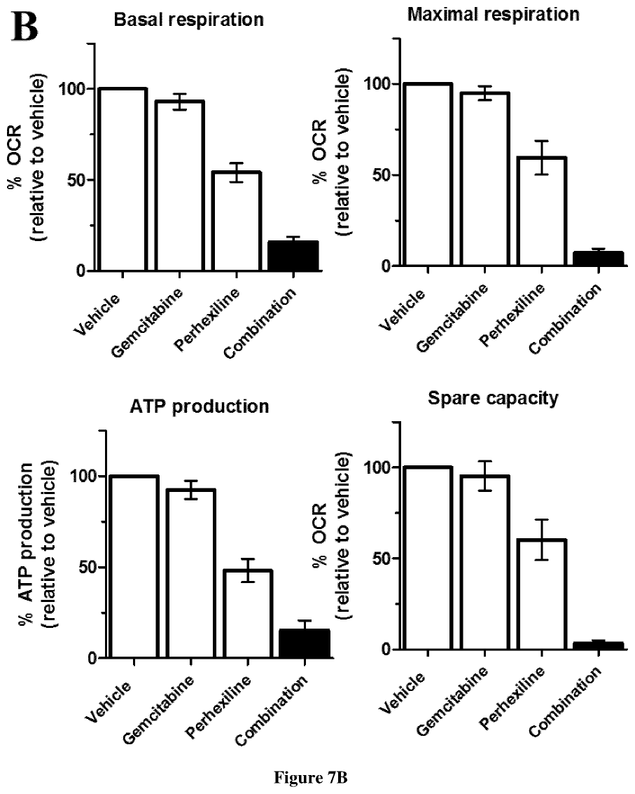

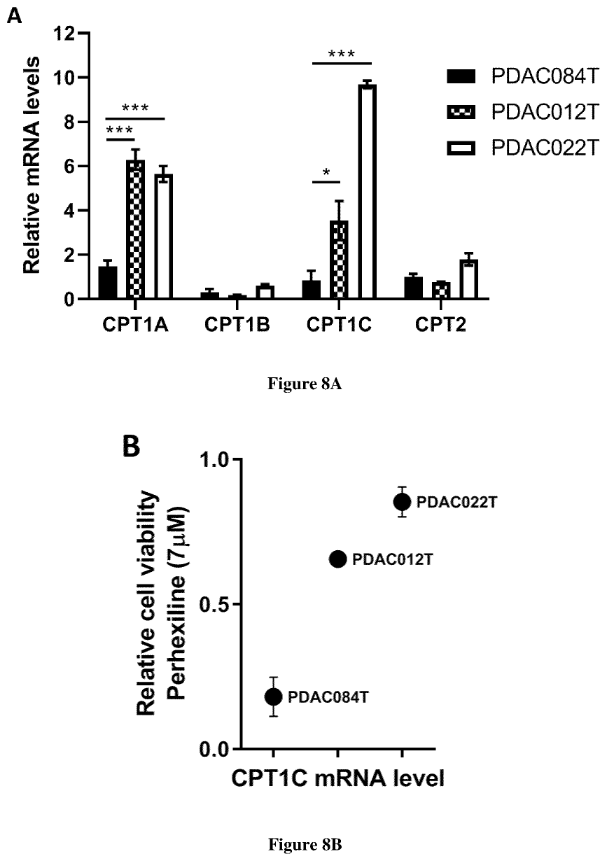

FIG. 8. CPT1C isoform as a key actor in the response to FAO targeting in PDAC. A. The high responder PDAC084T cells exhibit significantly lower mRNA levels of the CPT1A and CPT1C isoforms compared to the intermediate and low responder PDAC cells (PDAC012T and PDAC022T, respectively). The gene mRNA levels of CPT1 (A, B, C) and CPT2 isoforms were measured by RT-qPCR in three PDAC cells used in in vivo experiments. Data are presented as mean±SEM of three independent experiments performed in duplicates. P values from Student's t-test. B. The CPT1C mRNA levels correlate with response to FAO targeting with Perhexiline. The plot shows the mean of CPT1C mRNA levels in the previous Figure, compared with the relative cell viability with Perhexiline at 7 (see FIG. 1B).

TABLE 1
Basal Oxygen Consumption Rates (OCR) in 10 primary PDAC
cells from Patient-Derived Xenografts. The OCR (pmol/min/10
000 cells) is presented in decreasing order.

| Patient # | OCR |
|---|---|
| 84 | 102.29 |
| 27 | 60.96 |
| 12 | 46.13 |
| 21 | 44.54 |
| 03 | 44.4 |
| 82 | 31.73 |
| 01 | 24.5 |
| 22 | 23.51 |
| 32 | 23.27 |
| 85 | 15.52 |

### EXAMPLE

Material & Methods

Cell Culture

We used 21 PDAC primary cells obtained from Patient-Derived Xenografts (PDX) from the PaCaOmics cohort [53], in which patients were included under the Paoli-Calmettes Institute clinical trial number 2011-A01439-32. Consent forms of informed patients were collected and registered in a central database. PDAC cells were obtained and maintained in serum-free ductal media (SFDM) as described [54], and incubated at 37° C. in a 5% CO2 incubator. Cell lines were monthly tested for Mycoplasma contamination and found to be negative.

Real-Time Metabolic Analysis (XF Mito Fuel Flex Test)

We assessed the mitochondrial capacities of the 21 PDAC primary cells to oxidize the three main energetic sources: Glucose, Glutamine, and Fatty Acids.

Measurements were performed using the Seahorse Bioscience XFe24 Extracellular Flux Analyzer (Agilent). Sixteen hours before the assay, cells at exponential growth were seeded into Seahorse 24-well plates and cultured at 37° C. with 5% CO2. The number of seeded cells was optimized to ensure 70-80% confluence the day of analysis.

The Seahorse XF Mito Fuel Flex Test Kit was used to determine the Dependency of cells to oxidize the three energetic fuels. Culture medium was replaced by OXPHOS assay medium and the plate was pre-incubated for 1 h at 37° C. in a non-CO2 incubator. Inhibitors of mitochondrial pyruvate carrier (UK5099 2 μM), glutaminase (BPTES 3 μM), and CPT1a (Etomoxir 4 μM) were used to inhibit glycolysis, glutaminolysis, and FAO pathways, respectively. The rate of oxidation of each fuel was determined by measuring the oxygen consumption rate (OCR) by mitochondria in the presence or absence of fuel pathway inhibitors according to the manufacturers' instructions.

Dose-Response Viability Experiments In Vitro (Chemograms)

We tested the sensitivity of PDAC cells to three different drugs targeting the FAO pathway: Etomoxir, Perhexiline, and Trimetazidine (all provided by Sigma-Aldrich, Saint-Quentin Fallavier, France), and to the standard chemotherapy Gemcitabine (Gemzar, Eli Lilly & Co).

Cells were seeded in 96-well plates (5,000 cells per well) and 24 h later, the medium was supplemented with increasing concentrations of the drugs in triplicates. For the combination treatment, we used Perhexiline at 5 μM with increasing doses of Gemcitabine. Cell viability was determined 72 h later by Crystal violet viability assay, which is independent from cell metabolism. Briefly, cells were fixed in Glutaraldehyde 1%, washed twice with PBS, stained with crystal violet 0.1% for 10 min, and then washed three times with PBS. Crystals were solubilized in SDS 1%, and absorbance was measured at 600 nm using an Epoch-Biotek spectrophotometer.

To determine the synergistic effect in the combination treatments, we calculated the predicted values by multiplying the cell viability in the Gemcitabine and Perhexiline groups. Then, when the observed value is less than the predicted value, we consider it as a synergistic effect.

In Vivo Experiments

We assessed the FAO inhibition impact in vivo using the drug Perhexiline in combination with Gemcitabine. We performed subcutaneous xenografts in immunodeficient mice using four different primary PDAC cells. Recipient mice were 6-week-old female Swiss nude mice Crl:Nu(lco)-Foxn1nu purchased from Charles River, France. To obtain the xenografts, subcutaneous tumors from initial mouse donors were removed and finely minced with a scalpel. Then, 150 mg of tumor's pieces were mixed with 50 μl of Matrigel and implanted with a trocar (10 Gauge) in the subcutaneous space of anesthetized mice. Tumor volume was measured twice per week using a digital caliper and using the formula V=lenght×(width)/2. When tumor volume reached 200 mm³, mice were randomly assigned in a treatment scheme:

a) Gemcitabine: 120 mg/kg twice a week

b) Perhexiline: 5 mg/kg every other day

c) Combination treatment: Gemcitabine plus Perhexiline with the same described indications

d) Vehicle: PBS in the case of Gemcitabine treatment controls and 3% DMSO in PBS for combination treatment controls.

Mice were treated by intraperitoneal injection during one month, and mice in which tumor volume reached 1.5 cm³ during this period were ethically sacrificed and tumors removed.

All mice were kept under specific pathogen-free conditions and according to the current European regulation; the experimental protocol was approved by the Institutional Animal Care and Use Committee (#16711).

Flow Cytometry

Cells were seeded in 6-well plates in duplicates (150 000 cells per well) and the day after, the corresponding treatment was administered. We treated four PDAC cells with 7 μM of Perhexiline for 72 h (FIG. 5A), and in combination with 1 μM of Gemcitabine for 24 h for the PDAC084T cells. After the treatment period, cells were detached with Accutase, and resuspended in Annexin V-binding buffer (Biolegend). Cell death was determined using the Annexin V and Propidium Iodide (PI) staining (Biolegend), for apoptosis and necrosis, respectively. Ten thousand events per sample were acquired in a MAC SQuant-VYB (Miltenyi Biotec) and data analysis was done with the FlowJo software.

Ex Vivo Analysis (Immunohistochemistry and Histochemistry)

Tumor-bearing mice were sacrificed under treatment (middle-point of one-month treatment), and tumors fixed in 4% Paraformaldehyde, dehydrated and embedded in paraffin. Serial 4 μm sections were cut and stained with the Masson's Trichrome staining for collagen fibers detection, cleaved-Caspase 3 antibody (apoptosis), and ki67 (proliferation). Slides were scanned and images were captured using Calopix digital software. Quantification of stained areas were done with Fiji ImageJ.

Real-Time Metabolic Analysis (XF Cell Mito Stress Test)

We tested the impact of Gemcitabine, Gemcitabine, and the combination treatment (6 hours) on the mitochondrial respiration in PDAC084T cells. Measurements were performed using the Seahorse Bioscience XFe24 Extracellular Flux Analyzer (Agilent). Sixteen hours before the assay, cells at exponential growth were seeded into Seahorse 24-well plates and cultured at 37° C. with 5% CO₂. Six hours before the assay, the media was replaced with vehicle DMSO (0.01%), Gemcitabine (1 μM), Perhexiline (10 μM), or the combination of the two drugs. After 6 h treatment, the media was replaced with OXPHOS assay medium (DMEM without phenol red [Sigma-Aldrich reference D5030], 143 mM NaCl, 2 mM glutamine, 1 mM sodium pyruvate and 10 mM glucose, pH 7.4) and the plate was pre-incubated for 1 h at 37° C. in a non-CO₂ incubator. OCR was measured under basal conditions, and then after sequential injections of Oligomycin at 1 μM, carbonyl cyanide-p-trifluoromethox-yphenyl-hydrazon (FCCP, the concentration was optimized for each cell line), and 0.5 μM of Rotenone plus Antimycin A. Oligomycin is a respiratory Complex V inhibitor that allows to calculate ATP production by mitochondria, and FCCP is an uncoupling agent allowing the determination of the maximal respiration and the spare capacity. Finally, Rotenonte/Antimycin A are Complex I and III inhibitors, respectively that are injected to stop mitochondrial respiration enabling the calculation of the background (i.e., non-mitochondrial respiration driven by processes outside the mitochondria).

Gene Set Enrichment and RT-qPCR Analysis

We analyzed the RNA-sequencing data (RNA-seq) from five PDAC cells (#84, #82, #27, #12, #21) of the high and intermediate responder group to FAO targeting with Perhexiline; and three PDAC cells (#03, #32, #85) from the low responder group. Then, we used the KEGG database to determine the significantly upregulated or downregulated pathways between the two groups taking as reference the high/intermediate responder group.

Secondly, we measured the mRNA levels of the CPT1 (A, B, and C) and CPT2 isoforms by RT-qPCR in the PDAC cells used in the in vivo experiments (#84, #12, #22, #32). Briefly, cells were seed in 10 cm² petri dishes (one million cells per dish), and the day after, cells were subjected to treatments: vehicle DMSO (0.01%), Perhexiline 5 μM, Gemcitabine 1 μM, and the combination. 24 hours later, cells were detached and RNA was extracted using the RNeasy Mini kit (Qiagen) according to manufacturer's instruction. Next, RNA samples were subjected to reverse-transcription (Takara), and quantitative real-time PCR was performed in duplicates.

Results

Mitochondrial Respiration of Primary PDAC Cells Shows Dependency Towards Fatty Acids

Besides glucose, fatty acids and glutamine are main nutrients that feed the TCA cycle for cellular respiration producing ATP. Therefore, we addressed the dependency of mitochondrial respiration towards these three energetic fuels in 21 primary PDAC cells obtained from Patient-Derived Xenografts (PDX of the PaCaOmics biobank). Inhibitors of mitochondrial pyruvate carrier (UK5099), glutaminase (BPTES), and CPT1a (Etomoxir) were used to inhibit glycolysis, glutaminolysis, and FAO pathways, respectively. The cells' mitochondrial dependency on each of these fuel sources is determined by measuring the decrease in oxygen consumption rate (OCR) after addition of the specific inhibitor, followed by inhibition of the two alternative pathways.

Our results show that mitochondrial respiration depends mainly on fatty acids in the 21 primary PDAC cells (data not shown). Moreover, the percentage of dependency towards fatty acids was significantly higher than on glucose and glutamine. Indeed, 12 of the 21 cells exhibited a high dependence for this fuel (≥60%) and the other 9 a moderate dependency degree. Regarding glucose, the majority of PDAC cells showed a moderate reliance on this nutrient for respiration. Interestingly, a very low dependency on glutamine (average of 8%) was observed in all the PDAC cells. Therefore, in sharp contrast with several studies, our result takes the focus away from the glycolysis and glutaminolysis pathways, and brings to light the importance of FAO in PDAC. In conclusion, this finding places the FAO pathway as a novel vulnerability in PDAC.

PDAC Cells Exhibit Different Sensitivities to Pharmacological FAO Inhibitors and OXPHOS Rates Correlate with Response to FAO Targeting

The dependency of mitochondrial respiration on fatty acids in all the PDAC cells suggest that the deprivation of this nutrient could result in energetic stress promoting cancer cell elimination. Therefore, we evaluated the effect of FAO inhibition in vitro. We performed dose-response viability experiments treating PDAC cells for 72 h with three different drugs: Etomoxir, Perhexiline and Trimetazidine. Etomoxir and Perhexiline are inhibitors of the Carnitine Palmitoyl-Transferase 1 (CPT1), and Trimetazidine targets the last step of beta-oxidation.

Our results show that PDAC cells exhibit different sensitivity to the FAO inhibitors Etomoxir and Perhexiline (FIG. 1). FIGS. 1A and 1B show the relative cell viability of PDAC cells upon treatment with Etomoxir (62.5 μM) and Perhexiline (7 μM) compared with non-treated cells. These findings led us to pinpoint PDAC cells that respond efficiently to Etomoxir and Perhexiline, i.e. showing relative cell viability below the mean (0.75 for Etomoxir and 0.72 for Perhexiline) that we consider as “High responders”. Another group of cells show moderate response with values close to the mean, and finally some cells show low response (“Low responders”) with relative cell viability above the mean. Interestingly, no effect in any cell was seen upon Trimetazidine treatment compared with the non-treated cells (FIG. 1C).

Strikingly, no correlation was observed between response to pharmacological FAO inhibition and fatty acids dependency. By contrast, we found some link with basal mitochondrial respiration (basal oxygen consumption rate [OCR] shown in Table 1). Indeed, we observed a significant negative correlation between the OCR and the relative cell viability with Perhexiline (FIG. 2). This means that the High/moderate responders to Perhexiline correspond to the cells with High OCR (e.g. the highest OCR cell #84 is the most sensitive to Perhexiline), whereas the Low responders to Perhexiline are the ones with Low OCR activity. These observations were similar with the Etomoxir treatment, even if there is no statistical correlation. Altogether, our observations highlight the different sensitivities of PDAC cells to FAO inhibitors, and suggest that the basal OXPHOS status could predict the response to FAO targeting by Perhexiline.

The FAO Inhibitor Perhexiline in Combination with Gemcitabine is Synergistic Specifically in High Responder Cells

Furthermore, we wondered whether treating cells with a FAO inhibitor in combination with Gemcitabine could increase the efficacy of Gemcitabine. Based on our in vitro outcomes, we chose to use the potent FAO inhibitor Perhexiline with a low concentration (5 μM) that no impacts cell viability, or combined with increasing concentrations of Gemcitabine. For this, we treated four different PDAC cells with different sensitivities to Perhexiline (high, intermediate, and low responders). Interestingly, combining Perhexiline with Gemcitabine specifically sensitizes the High and intermediate responders cells PDAC084T and PDAC012T, showing a strong synergistic effect (FIG. 3A-B), which is not the case for the low responder cells PDAC022T and PDAC032T (FIG. 3C-D).

FAO Inhibition with Perhexiline in Combination with Gemcitabine Induces Complete Tumor Regression in a High Responder PDAC Xenograft

We then addressed the question of the impact of FAO inhibition on chemotherapeutic response in vivo. We performed subcutaneous xenografts in immunodeficient mice using the same four different primary PDAC cells treated with the combination in vitro. We selected the PDAC cells regarding the in vitro sensitivity to Perhexiline and the combination with Gemcitabine (FIGS. 1B and 3), and the basal OCR rate (Table 1). Accordingly, we worked with a high responder cell with high OXPHOS rate (PDAC084T), a moderate responder with intermediate OXPHOS rate (PDAC012T), and low responder cells with low OXPHOS rate (PDAC022T and PDAC032T). Tumor-bearing mice were treated during one month using Gemcitabine alone or in combination with Perhexiline.

FIG. 4 shows the progressive tumor growth in the four PDAC xenografts, upon treatment with Gemcitabine, Perhexiline, or the combination Gemcitabine+Perhexiline. Overall, we observed that Perhexiline per se doesn't have any impact on tumor growth compared with vehicle-injected mice. In both high and intermediate OXPHOS contexts (FIG. 4A-C), treatment with Gemcitabine alone is arresting the tumor growth, suggesting a cytostatic effect. Excitingly, in the High responder xenograft (PDAC084T), the co-administration of Perhexiline and Gemcitabine significantly potentiates the efficacy of Gemcitabine, and more importantly, induces a complete tumoral regression after one-month treatment (FIG. 4A). In the intermediate responder xenograft (PDAC012T), the mice treated with the combination showed a higher tumor growth inhibition compared with the chemotherapy alone (FIG. 4B). Finally, tumors from the low responder xenograft PDAC032T show a high sensitivity to Gemcitabine alone which induces tumor regression. No impact was observed with the combination treatment compared with Gemcitabine alone, in both low responder models PDAC032T and PDAC022T (FIG. 4C, D). Altogether, we demonstrate that targeting FAO using Perhexiline enhances the antitumoral activity of Gemcitabine in two of our pre-clinical models. Moreover, combining Perhexiline with Gemcitabine results in a complete tumor regression in the high responder PDAC xenograft. This means that applying this therapeutic strategy could notably improve the prognosis of PDAC in a subset of patients.

The FAO Inhibitor Perhexiline Enhances the Antitumor Activity of Chemotherapy by Inducing Apoptosis and Energetic Stress in Pancreatic Cancer

Based on the above outcomes, we further investigated the mechanism of cooperation between Perhexiline and Gemcitabine underlying the complete tumor regression in the high responder PDAC xenograft (PDAC084T). For that, we performed cell death assays demonstrating that Perhexiline is an apoptotic inductor, and we clearly illustrate the different responses in the four PDAC cells in terms of cell death (FIG. 5A). Moreover, the combination of Perhexiline plus Gemcitabine in PDAC084T cells results in an increase in cell death by apoptosis with the consequent decrease in cell viability (FIG. 5B).

More importantly, we investigated the mechanism of cooperation between Perhexiline and Gemcitabine in vivo. Using the same mouse models of PDAC xenografts, we excised the tumors from mice under treatment (middlepoint of one-month treatment) in the PDAC084T xenograft. Next, we performed immunohistochemistry analysis for apoptosis (cleaved-Caspase 3) and proliferation (ki67) markers, as well as histochemistry with the Masson's Trichrome staining (to detect fibrosis). Similarly to the cellular investigation, we observe that the PDAC084T tumors treated with the combination therapy show a higher staining for cleaved-Caspase 3 in comparison with Gemcitabine alone (FIG. 6A), suggesting that Perhexiline increases the Gemcitabine-induced apoptosis in vivo, having as a result a complete tumor regression. In contrast, no difference for ki67 (proliferation) is observed between Gemcitabine or combination treatment groups (FIG. 6B). However, the tumors from mice treated with the combination, show larger fibrotic areas compared with the other mice groups (FIG. 6C).

Finally, to continue deciphering the mechanism of cooperation between Perhexiline and Gemcitabine in PDAC084T, we determined the impact of Gemcitabine, Perhexiline, and the combination, on the mitochondrial respiration (Seahorse metabolic analyzer). FIG. 7A clearly illustrates that the FAO inhibitor Perhexiline is able to decrease mitochondrial respiration in about 50%. More importantly, the combination of Gemcitabine and Perhexiline drastically decreases basal and maximal mitochondrial respiration, ATP production, and spare respiratory capacity, leading cells to an energetic crisis (FIG. 7B).

CPT1C Isoform as a Key Actor in the Response to FAO Targeting in PDAC

To decipher the key molecular actors related to the response to FAO targeting combined with chemotherapy, we performed a transcriptomic analysis (RNA sequencing and RT-qPCR). For the first analysis, we analyzed the RNA-sequencing data (RNA-seq) from five PDAC cells belonging to the high and intermediate responder group, and 3 PDAC cells from the low responder group. In this data, we used the KEGG database to determine the significantly upregulated or downregulated pathways between the two groups. Interestingly, our results point to the CPT1C isoform, which is significantly downregulated in the high responder group. Then, for the second analysis, we measured the mRNA levels of the CPT1 (A, B, and C) and CPT2 isoforms by RT-qPCR in the PDAC cells used in the in vivo experiments. Consistently, we found that the mRNA level of the CPT1C isoform in the PDAC084T cells is significantly lower in comparison with the other cells and that the CPT1C mRNA levels correlate with the response to Perhexiline treatment (FIG. 8A-B).

Taking together this data, our molecular analysis points to the CPT1C enzyme as a key actor in the mechanism of cooperation between Perhexiline and Gemcitabine to induce complete pancreatic cancer regression in the PDAC084T xenograft. Currently, we are working on deciphering how the different treatments impact on the CPT1C gene expression, and more importantly, we are working on the manipulation of the CPT1C mRNA levels in PDAC084T. Our hypothesis proposes that the low mRNA level of CPT1C in PDAC084T is responsible for the complete tumor regression observed, and that by increasing these levels, the combination therapy will lose its efficacy.

### CONCLUSION

In conclusion, the work of the inventors demonstrates that Fatty Acid Oxidation (FAO) inhibitors could be used for the treatment of pancreatic cancer (Pancreatic Ductal AdenoCarcinoma). CPT1, a key enzyme in the FAO pathway, can be pharmacologically targeted by drugs like Etomoxir and Perhexiline. Moreover, in this work, the inventors used Perhexiline and showed enhancement of the antitumoral effect of chemotherapy (Gemcitabine), resulting in a complete tumoral regression in preclinical assays for a subset of patients (“high responder”, namely “high OXPHOS”). Thus, such combinations could also be used in High OXPHOS patients. Further, this work provides the mechanism of cooperation between Perhexiline and Gemcitabine, and points to the CPT1C isoform as a potential biomarker that predict the response of patients to FAO targeting.

### REFERENCES

Throughout this application, various references describe the state of the art to which this invention pertains. The disclosures of these references are hereby incorporated by reference into the present disclosure.
1. Bray F, Ferlay J, Soerjomataram I, Siegel R L, Torre L A, Jemal A. Global cancer statistics 2018: GLOBOCAN estimates of incidence and mortality worldwide for 36 cancers in 185 countries. CA Cancer J Clin. 2018; 68(6):394-424. doi: 10.3322/caac.21492.
2. McGuigan A to al. Pancreatic cancer: A review of clinical diagnosis, epidemiology, treatment and outcomes. World J Gastroenterol. 2018; 24(43):4846-4861. doi: 10.3748/wjg.v24.i43.4846.
3. Ilic M, Ilic I. Epidemiology of pancreatic cancer. World J Gastroenterol. 2016; 22:9694-9705.
4. Saad A M, Turk T, Al-Husseini M J, Abdel-Rahman O. Trends in pancreatic adenocarcinoma incidence and mortality in the United States in the last four decades; a SEER-based study. BMC Cancer. 2018; 18:688.
5. Siegel R L, Miller K D, Jemal A. Cancer Statistics, 2017. CA Cancer J Clin. 2017; 67:7-30.
6. Rahib L net al. Projecting cancer incidence and deaths to 2030: the unexpected burden of thyroid, liver, and pancreas cancers in the United States. Cancer Res. 2014; 74:2913-2921.
7. Roser, M. & Ritchie, H. Cancer. Our World in Data https://ourworldindata.org/cancer (2018).
8. Rawla P, Sunkara T, Gaduputi V. Epidemiology of Pancreatic Cancer: Global Trends, Etiology and Risk Factors. World J Oncol. 2019; 10(1):10-27. doi:10.14740/wjon1166.
9. Conroy, T. et al. FOLFIRINOX versus gemcitabine for metastatic pancreatic cancer. N. Engl. J. Med. 2011; 364, 1817-1825.
10. Von Hoff, D. D. et al. Increased survival in pancreatic cancer with nab-paclitaxel plus gemcitabine. N. Engl. J. Med. 2013; 369, 1691-1703.
11. Conroy, T. et al. FOLFIRINOX or gemcitabine as adjuvant therapy for pancreatic cancer. N. Engl. J. Med. 2018; 379(25):2395-2406. doi: 10.1056/NEJMoa1809775.
12. Nevala-Plagemann, C., Hidalgo, M. & Garrido-Laguna, I. From state-of-the-art treatments to novel therapies for advanced-stage pancreatic cancer. Nat Rev Clin Oncol (2019). doi:10.1038/s41571-019-0281-6.
13. Halbrook C J, Lyssiotis C A. Employing Metabolism to Improve the Diagnosis and Treatment of Pancreatic Cancer. Cancer Cell. 2017; 31(1):5-19. doi: 10.1016/j.ccell.2016.12.006.
14. Vander Heiden, M. G. Targeting cancer metabolism: a therapeutic window opens. Nat. Rev. Drug Discov. 2011; 10, 671-684.
15. Rajeshkumar N V, et al. Treatment of Pancreatic Cancer Patient-Derived Xenograft Panel with Metabolic Inhibitors Reveals Efficacy of Phenformin. Clin Cancer Res. 2017; 23(18):5639-5647. doi: 10.1158/1078-0432.CCR-17-1115.
16. Daemen A, et al. Metabolite profiling stratifies pancreatic ductal adenocarcinomas into subtypes with distinct sensitivities to metabolic inhibitors. Proc Natl Acad Sci USA. 2015; 112(32): E4410-E4417. doi: 10.1073/pnas.1501605112.
17. Warburg, O. On the origin of cancer Cells. Science. 1956; 123(3191):309-14.
18. Hanahan, D., and Weinberg, R. A. Hallmarks of cancer: the next generation. Cell.

2011; 144, 646-674.
19. Biancur D E, Kimmelman A C. The plasticity of pancreatic cancer metabolism in tumor progression and therapeutic resistance. Biochim Biophys Acta Rev Cancer. 2018; 1870(1):67-75. doi:10.1016/j.bbcan.2018.04.011.
20. Cannon A, Thompson C, Hall B R, Jain M, Kumar S, Batra S K. Desmoplasia in pancreatic ductal adenocarcinoma: insight into pathological function and therapeutic potential. Genes Cancer. 2018; 9(3-4):78-86. doi:10.18632/genesandcancer.171.
21. Vaziri-Gohar A, Zarei M, Brody J R, Winter J M. Metabolic Dependencies in Pancreatic Cancer. Front Oncol. 2018; 8:617. doi:10.3389/fonc.2018.00617.
22. Vyas S, Zaganjor E, Haigis M C. Mitochondria and Cancer. Cell. 2016; 166(3):555-566. doi:10.1016/j.cell.2016.07.002.
23. Emmings E, Mullany S, Chang Z, Landen C N Jr, Linder S, Bazzaro M. Targeting Mitochondria for Treatment of Chemoresistant Ovarian Cancer. Int J Mol Sci. 2019; 20(1):229. doi:10.3390/ijms20010229.
24. Dijk S N, Protasoni M, Elpidorou M. et al. Mitochondria as target to inhibit proliferation and induce apoptosis of cancer cells: the effects of doxycycline and gemcitabine. Sci Rep. 2020; 10, 4363.
25. Ashton T M, McKenna W G, Kunz-Schughart L A, et al. Oxidative Phosphorylation as an Emerging Target in Cancer Therapy. Clin Cancer Res 2018; 24(11):2482-2490.
26. Alistar A. et al. Safety and tolerability of the first-in-class agent CPI-613 in combination with modified FOLFIRINOX in patients with metastatic pancreatic cancer: a single-centre, open-label, dose-escalation, phase 1 trial. Lancet Oncol. 2017; 18, 770-778. doi: 10.1016/S1470-2045(17)30314-5.
27. Nguyen C, Pandey S. Exploiting Mitochondrial Vulnerabilities to Trigger Apoptosis Selectively in Cancer Cells. Cancers (Basel). 2019; 11(7):916. doi:10.3390/cancers11070916.
28. Farge T, Saland E, de Toni F, et al. Chemotherapy-Resistant Human Acute Myeloid Leukemia Cells Are Not Enriched for Leukemic Stem Cells but Require Oxidative Metabolism. Cancer Discov 2017; 7(7):716-735.
29. Gentric G, Kieffer Y, Mieulet V, et al. PML-Regulated Mitochondrial Metabolism Enhances Chemosensitivity in Human Ovarian Cancers. Cell Metab 2019; 29(1):156-173 e10.
30. Tae Gyu Choi and Sung Soo Kim. Physiological Functions of Mitochondrial Reactive Oxygen Species [Online First], IntechOpen, 2019. DOI: 10.5772/intechopen.88386. Available from: https://www.intechopen.com/online-first/physiological-functions-of-mitochondrial-reactive-oxygen-species.
31. Cocetta V, Ragazzi E, Montopoli M. Mitochondrial Involvement in Cisplatin Resistance. Int J Mol Sci. 2019; 20(14):3384. doi:10.3390/ijms20143384.
32. Tan A S, Baty J W, Dong L F, et al. Mitochondrial genome acquisition restores respiratory function and tumorigenic potential of cancer cells without mitochondrial DNA. Cell Metab 2015; 21:81-94.
33. Park S. Y., Chang I., Kim J. Y., Kang S. W., Park S. H., Singh K., Lee M. S. Resistance of mitochondrial DNA-depleted cells against cell death role of mitochondrial superoxide dismutase. J. Biol. Chem. 2004; 279:7512-7520. doi: 10.1074/jbc.M307677200.
34. Montopoli M. et al. “Metabolic reprogramming” in ovarian cancer cells resistant to cisplatin. Curr. Cancer Drug Targets. 2011; 11:226-235. doi: 10.2174/156800911794328501.
35. Mei H., Sun S., Bai Y., Chen Y., Chai R., Li H. Reduced mtDNA copy number increases the sensitivity of tumor cells to chemotherapeutic drugs. Cell Death Dis. 2015;

6:e1710. doi: 10.1038/cddis.2015.78.
36. Wang M, Lu X, Dong X, Hao F, Liu Z, Ni G, Chen D. pERK1/2 silencing sensitizes pancreatic cancer BXPC-3 cell to gemcitabine-induced apoptosis via regulating Bax and Bc1-2 expression. World J Surg Oncol. 2015; 13:66. doi: 10.1186/s12957-015-0451-7.
37. Carracedo A, Cantley L C, Pandolfi P P. Cancer metabolism: fatty acid oxidation in the limelight. Nature Reviews Cancer 2013; (13): 227-232.
38. Lionetti V, Stanley W, Recchia F. Modulating fatty acid oxidation in heart failure. Cardiovascular research. 2011; 90, 202-209.
39. Holubarsch, C. J. et al. A double-blind randomized multicentre clinical trial to evaluate the efficacy and safety of two doses of etomoxir in comparison with placebo in patients with moderate congestive heart failure: the ERGO (etomoxir for the recovery of glucose oxidation) study. Clin. Sci. (Lond.) 2007; 113, 205-212.
40. Kantor, P. F. et al. The antianginal drug trimetazidine shifts cardiac energy metabolism from fatty acid oxidation to glucose oxidation by inhibiting mitochondrial longchain 3-ketoacyl coenzyme A thiolase. Circ. Res. 2000; 86, 580-588.
41. Nash, D. T. & Nash, S. D. Ranolazine for chronic stable angina. Lancet. 2008; 372, 1335-1341.
42. Kolwicz S C Jr, Purohit S, Tian R. Cardiac metabolism and its interactions with contraction, growth, and survival of cardiomyocytes. Circulation research. 2013; 113(5):603-16.
43. Zachary T, et al. Antioxidant and Oncogene Rescue of Metabolic Defects Caused by Loss of Matrix Attachment. Nature. 2009; 461(7260): 109-113.
44. Carracedo, A. et al. A metabolic prosurvival role for PML in breast cancer. J. Clin. Invest. 2012; 122, 3088-3100.
45. Zaugg, K. et al. Carnitine palmitoyltransferase 1C promotes cell survival and tumor growth under conditions of metabolic stress. Genes Dev. 2011; 25, 1041-1051.
46. Samudio, I. et al. Pharmacologic inhibition of fatty acid oxidation sensitizes human leukemia cells to apoptosis induction. J. Clin. Invest. 2010; 120, 142-156.
47. Camarda R, et al. Inhibition of fatty acid oxidation as a therapy for MYC-overexpressing triple negative breast cancer. Nature medicine. 2016; 22:427-432.
48. Schlaepfer I R et al. Lipid catabolism via CPT1 as a therapeutic target for prostate cancer. Mol Cancer Ther. 2014; (10):2361-71.
49. Schlaepfer I R et al. Inhibition of Lipid Oxidation Increases Glucose Metabolism and Enhances 2-Deoxy-2-[(18)F]Fluoro-D-Glucose Uptake in Prostate Cancer Mouse Xenografts.

Mol Imaging Biol. 2015; 17(4):529-38.
50. Tirado-Velez J M, Joumady I, Sáez-Benito A, Cózar-Castellano I, Perdomo G. Inhibition of fatty acid metabolism reduces human myeloma cells proliferation. PLoS One. 2012; 7(9):e46484.
51. Vella S, Penna I, Longo L, Pioggia G, Garbati P, Florio T, Rossi F, Pagano A. Perhexiline maleate enhances antitumor efficacy of cisplatin in neuroblastoma by inducing overexpression of NDM29 ncRNA. Scientific reports. 2015; 5:18144.
52. Ren X R, Wang J, Osada T, Mook R A Jr, Morse M A, Barak L S, Lyerly H K, Chen W. Perhexiline promotes HER3 ablation through receptor internalization and inhibits tumor growth. Breast cancer research. 2015; 17:20.
53. Gayet O et al. A subgroup of pancreatic adenocarcinoma is sensitive to the 5-aza-dC DNA methyltransferase inhibitor. Oncotarget 2015; 6:746-54.
54. Schreiber F S et al. Successful growth and characterization of mouse pancreatic ductal cells: functional properties of the Ki-RAS (G12V) oncogene. Gastroenterology. 2004; 127:250-260.

Yibao Ma, Sarah M. Temkin, Adam M. Hawkridge, Chunqing Guo, Wei Wang, Xiang-Yang Wang, Xianjun Fang. Fatty acid oxidation: An emerging facet of metabolic transformation in cancer. Cancer Letters 435 (2018) 92-100.

Tonazzi, A, Nicola G, Lara C, De Palma A, Indiveri C. Nitric Oxide Inhibits the Mitochondrial Carnitine/Acylcarnitine Carrier through Reversible S-Nitrosylation of Cysteine 136. Biochimica et Biophysica Acta (BBA)—Bioenergetics. 2017: 475-82.

Chegary M, Te Brinke H, Doolaard M, et al. Characterization of L-aminocarnitine, an inhibitor of fatty acid oxidation. Mol Genet Metab. 2008; 93(4):403-410.

1. A method of treating pancreatic cancer in a patient in need thereof, comprising,
administering to the patient a therapeutically effective amount of an inhibitor of fatty acid oxidation (FAO).

2. The method of claim 1, wherein the inhibitor of FAO is administered simultaneously, separately or sequentially with a therapeutic compound used to treat pancreatic cancer.

3. The method according to claim 1 wherein the patient has a high OXPHOS profile.

4. The method according to according to claim 1, wherein the inhibitor of FAO is an inhibitor of CPT1, CACT, CPT2 or 3-KAT.

5. The method according to claim 4 wherein the inhibitor of FAO is an inhibitor of CPT1.

6. The method according to claim 1 wherein the inhibitor of CPT1 is Etomoxir, Perhexiline, Oxfenicine, Methyl palmoxirate, S-15176, Metoprolol, or amiodarone.

7. The method according to claim 2 wherein the therapeutic compound used to treat pancreatic cancer is gemcitabine, 5-fluorouracil (5-FU), Capecitabine, oxiplatin, cisplatin, irinotecan, or Nab-Paclitaxel, or a combination of folinic acid, 5-FU, irinotecan and oxaliplatin.

8. A therapeutic composition comprising an inhibitor of fatty acid oxidation (FAO) formulated for use in the treatment of pancreatic cancer in a patient in need thereof.

9. (canceled)

10. An in vitro method for predicting FAO inhibitor response of a patient in need thereof and treating the patient, comprising: i) determining, in a sample obtained from the patient, an expression level of a CPT1C isoform; ii) determining that the expression level determined at step i) is lower than a reference value, and iii) treating the patient determined to have an expression level that is lower than the reference value with an FAO inhibitor.

11. An in vitro method for monitoring FAO inhibitor treatment in a subject in need thereof and then treating the subject, comprising the steps of i) determining, in a sample obtained from said subject after treating the subject with the FAO inhibitor, an expression level of CPT1C isoform; ii) determining that the expression level determined at step i) is lower than a reference value, and iii) treating the subject with the FAO inhibitor.

12. The in vitro method according to claim 11, wherein the reference value is the expression level of CPT1C determined in samples obtained from the subject before FAO inhibitor treatment.

13. A method for treating pancreatic cancer in a patient in need thereof comprising i) determining, in a sample obtained from the patient, an expression level of a CPT1C isoform; and ii) administering a therapeutically effective amount of an FAO inhibitor to the patient determined to have an expression level of CPT1C lower than a reference value.
